(19) United States
(12) Patent Application Publication
(10) Pub. No.: US 2007/0128658 A1
(43) Pub. Date: Jun. 7, 2007

(54) FLUORESCENT DYES, METHODS AND USES THEREOF

(76) Inventors: Helen E. Blackwell, Middleton, WI (US); Matthew D. Bowman, Madison, WI (US)

Correspondence Address: GREENLEE WINNER AND SULLIVAN P C, 4875 PEARL EAST CIRCLE, SUITE 200, BOULDER, CO, 80301, US

(21) Appl. No.: 11/559,510

(22) Filed: Nov. 14, 2006

Related U.S. Application Data
(60) Provisional application No. 60/597,159, filed on Nov. 14, 2005.

(51) Int. Cl. G01N 33/53; C07D 405/02; C07D 487/02

(52) U.S. Cl. 435/7.1; 544/279; 546/268.1; 546/284.1; 546/286; 544/329

(57) ABSTRACT

The present invention provides a method for identifying and characterizing candidate fluorescent molecules. This method involves the generation of spatially addressed arrays of heterocyclic compounds which are candidate fluorescent molecules. The spatially addressed array can be used to identify fluorescent molecules among the candidate molecules. The spatially addressed array can be used to determine the optical characteristics of one more candidate molecules. The invention also provides methods for making such arrays of heterocyclic compounds. In a specific embodiment, synthetic methods are provided for synthesis of triarylpyridines, particularly unsymmetric triarylpyridines. n preferred embodiments, the present invention provides novel heterocyclic fluorescent compounds having deazalumazine, cyanopyridine or pyrimidine cores. Heterocyclic fluorescent compounds of this invention are useful as dyes to label molecules such as nucleic acids, peptides and proteins, and particularly antibodies. In other embodiments, the novel heterocyclic fluorescent compounds are useful as dyes to label cellular compartments and organelles. The novel heterocyclic fluorescent compounds can also be used as sensors or indicators of specific metal ions, chemical warfare agents and pH.

### CROSS-REFERENCE TO RELATED APPLICATIONS

This application claims priority to U.S. provisional application Ser. No. 60/597,159, filed Nov. 14, 2005, which is incorporated by reference herein in its entirety.

### STATEMENT REGARDING U.S. GOVERNMENT FUNDING

This work was funded at least in part with U.S. government funding through the National Science Foundation grant CHE-0449959. The United States government has certain rights in this invention.

### BACKGROUND OF THE INVENTION

The invention generally relates to heterocyclic fluorescent compounds, methods of making such compounds and assessing their fluorescent properties and methods of using such compounds

Fluorescent probes have become essential tools for the exploration of biological systems. Tsien, R. Y. In Fluorescent and Photochemical Probes of Dynamic Biochemical Signals Inside Living Cells; Czarnik, A. W. Ed.; American Chemical Society: Washington, D.C., 1993: pp 130-146; Lacowicz, J. R. Principles of Fluorescence Spectroscopy. New York: Kluwer Academic/Plenum Publishers, 1999. Techniques such as fluorescence polarization and fluorescence resonance energy transfer have been used to expand significantly the understanding of biomolecular interactions in vitro and in vivo. Yan, Y.; Marriott, G. Curr. Opin. Chem. Biol. 2003, 7, 635-640; Hahn, K.; Toutchkine, A. Curr. Opin. Cell. Biol. 2002, 14, 167-172. Likewise, the emergence of environmentally-sensitive fluorophores has enabled real-time imaging of complex cellular processes. Toutchkine, A.; Kraynov, V.; Hahn, K. J. Am. Chem. Soc. 2003, 125, 4132-4145. As the applications for fluorescent probes continue to increase, so does the need for new small molecule dyes with diverse spectral and physical properties.

Recently, combinatorial chemistry methods have been reported to expedite the identification of new dye classes. See e.g., Jiao, G. S., et al., Org. Lett. 2003, 5, 3675-3677; Schiedel, M. et al., Angew. Chem. Int. Ed. 2001, 40, 4677-4680; Li, Q., et al., Angew. Chem. Int. Ed. 2004, 43, 6331-6335; Rosania, G. R., et al., J. Am. Chem. Soc. 2003, 125, 1130-1131.

Small molecule macroarrays have been synthesized based on microwave SPOT-synthesis. Frank, R. Tetrahedron 1992, 48, 9217-9232; Bowman, M. D. et al. Organ. Lett. 2004, 6, 2019-2022; Lin, Q. et al. Org. Lett. 2005, 7, 4455-4458. The present invention provides methods for array synthesis that are useful in the identification and characterization of fluorescent molecules.

### SUMMARY OF THE INVENTION

The invention relates to methods for identifying and characterizing the properties of fluorescent molecules. The methods are based on the use of combinatorial synthetic methods to generate an array comprising a plurality of surface-bound candidate fluorescent molecules. Structurally distinct candidates are bonded to distinct known locations (e.g., spots) on a surface to form a spatially-addressed array of candidates. Emission from the spatially-addressed surface-bound array of candidate molecules can be employed to identify which candidates are fluorescent. Once candidates are identified as fluorescent, the spectral properties of any fluoresecent molecules can be determined by conventional methods. Alternatively, emission from the spatially-addressed surface-bound array of candidates can be measured to determine spectral properties of one or more fluorescent molecules in the array. Spectral properties that can be assessed include excitation wavelength distribution, emission wavelength distribution, Stroke's Shift, quantum yield or photodecomposition yield.

Candidate fluorescent molecules are synthesized in an array bound to a surface. The candidates are typically linked to the surface by a linker group. Linker groups include those that are cleavable, e.g., chemically or photochemically, so that candidate fluorescent molecules can be removed from the surface, preferably without destruction of the fluorescent moiety.

More specifically, the invention provides a method for identifying fluorescent molecules which comprises the steps of: providing an array of surface-bound spatially-addressable candidate fluorescent molecules on a unitary substrate; illuminating the array of candidate fluorescent molecules on the unitary substrate with light of selected wavelength to excite fluorescence of the array of candidate fluorescent molecules bound to the unitary substrate; and measuring the spatial distribution of fluorescence emitted from the array to identify fluorescent molecules in the array. Emission will be associated only with locations on the array to which fluorescent molecules are bound. The spatial distribution of emission from an array of candidates will also provide a measure of relative emission intensities. In one embodiment, an array of candidate fluorescent molecules is interrogated with UV light and emission from the array is visual detected or can be photographed. Interrogation and detection of an array can be automated, if desired, and any detector that allows the spatial distribution of emission from the array to be measured can be employed.

The spatially-addressed array of candidate fluorescent molecules is synthesized by providing a first spatially-addressed array of reactive compounds which are bound to a unitary substrate. The reactive compounds on the array are reacted, typically in a one step reaction, (e.g., a condensation reaction) with one or more reactive compounds, reagents or both which are not bound to the surface to form a second array of candidate molecules bound to the surface. The reactive compounds of the first array comprise a set of structurally related molecules having the same reactive moiety and differing structurally from each other in ways that preferably do not significantly decrease the reactivity of the reactive moiety shared by the members of the array. In specific embodiments, the candidate fluorescent molecules are heterocyclic molecules, more specifically the candidate fluorescent molecules are N-containing heterocycles, such as pyridines and pyrimidines.

In specific embodiments, the reactive moiety of the first array is an alpha-beta unsaturated ketone, and more specifically the reactive molecules of the first array are chalcones. In a specific embodiment, the non-surface bound reactant and/or reagent is a chemical compound which comprised two nucleophiles which react with the alpha-beta unsaturated ketone to form a heterocyclic ring. Preferably, one of the nucleophiles is —NH₂.

In a first embodiment, the non-surface bound reactant and/or reagent is 2-aminocrotylnitrile, and the candidates synthesized are cyanopyridines. In a second embodiment, the non-surface-bound reactant and/or reagent is an aminouracil in the presence of base and the candidates synthesized are deazalumazines. In a third embodiment, the non-surface bound reactant or reagent is an amidine salt and the candidates synthesized are pyrimidines or dihydropyrimidines. In a more specific aspect of this third embodiment, the non-surface bound reactant and/or reagent is a guanidine salt and the candidates are 2-amino pyrimidines or dihydropyrimidines. In another specific aspect of this third embodiment, the non-surface-bound reactant or reagent is an acetamidine salt or a benzamidine salt and the candidates are 2-methyl pyrimidines or dihydropyrimidines or 2-amino pyrimidines or dihydropyrimidines.

In a fourth embodiment, the non-surface-bound reactant and/or reagent is hydrazine hydrate and the surface-bound candidates are diaryl pyrazoles. In a fifth embodiment, the non-surface-bound reactant and/or reagent is an aryl hydrazine and the surface-bound candidates are diaryl N-arylpyrazoles. In a sixth embodiment, the non-surface-bound reactant and/or reagent is an aminocyclohexenone and the surface-bound candidates are diaryl pyridine derivatives. In a seventh embodiment, the non-surface-bound reactant, reagent or both is an aminobenzimidazole and the surface-bound candidates are diaryl tetrahydropyrimidines. In a eighth embodiment, the non-surface-bound reactant, reagent or both is a mixture of an isatin and an amino acid and the surface-bound candidates are isatin-chalone adducts.

In another specific embodiment, the reactive moiety of the reactive compounds of the first array is a diketone moiety and more specifically the reactive molecules of the first array are triary-1,5-pentane diones. In a specific embodiment, the non-surface-bound reactant is ammonium acetate and the surface-bound candidates are triarylpyridines which may be symmetric (where two aryl groups are the same) or unsymmetric (where all three aryl groups are different).

In certain embodiments, the heterocyclic candidate arrays are prepared by reaction of surface-bound arrays of alpha-beta unsaturated ketones, such as chalcones. Spatially-addressed arrays of chalcones can be prepared by reaction of a set of surface-bound acetophenones with a set of derivatized benzaldehydes. For example a 4×4 member array is prepared by linking 4 different acetophenones to a surface in aspatially-addressable array of 16 spots (4 spots of each acetophenone). Each of the 4 different benzaldehdyes is then reacted with one of the 4 array spots of each acetophenone to give a total array of 16 different chalcones.

In certain embodiments, the candidate arrays are prepared by further reaction of surface-bound diketones, such as diaryl-1,5-pentanediones. An array of diaryl-1,5-pentanediones can be prepared by reaction of a set of acetophenone derivatives with surface-bound arrays of chalcones. More specifically, each one of the non-surface bound acetophenones of a set of acetophenones is reacted with an array of chalcones. For example, a 16-member array of chalcones reacted with each of four different acetophenones would result in a 64-member array of diaryl-1,5-pentanediones.

In specific embodiments, one or more reactions employed for preparation of arrays of compounds herein are carried out by providing microwave electromagnetic radiation to the array during reaction.

The unitary substrate carrying the arrays of this invention is preferably a planar substrate and is more preferably a planar cellulose substrate. As noted above the substrate can be derivatized with acid cleavable or photocleavable linkers to which the component compounds of the arrays are bonded. The methods of this invention are particularly useful when practiced with macroarrays. However, the methods can be practiced employing microarrays.

In one aspect the invention provides a method for in situ synthesis of surface-bound arrays of heterocyclic molecules which can be screened to identify fluorescent molecules in the array. In a second aspect, the invention provides a method to identify fluorescent molecules in such arrays. In a related aspect, the invention provides a method for measuring optical properties of fluorescent molecules in an array. The invention further provides surface-bound arrays of candidate molecules. In specific embodiments, the invention provides arrays of such candidates having two or more surface-bound spatially-addressed candidates. Preferably the arrays, contain at least 4 candidates and preferably 16 or more candidates.

In other aspects, the invention provides fluorescent molecules which are identified by the screening method herein or which result from structure-function information derived from such screening methods. In specific embodiments, the invention provides fluorescent compounds with a heterocyclic core of formula I: 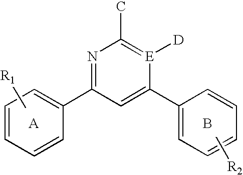
where:
the A and B rings are aryl rings,
C, D and E are defined in the alternative as:
(1) C is an aryl ring having an R₃ substituent, E is carbon and D is a hydrogen or a cyano group;
(2) E is carbon and C and D together form a 5- or 6-member optionally-substituted carbon ring in which one or two ring carbons are replaced with nitrogens; or
(3) E is nitrogen, D is absent and C is an alky or aryl ring;
R₁, R₂ and R₃ represent one or more than one substituent on the indicated ring, wherein each R₁, R₂ and R₃, independently of other R₁, R₂ and R₃ groups, are selected from hydrogen, —NO₂, —CN, halogen, alkyl, alkenyl, alkynyl, phenyl, —OH, —OR, —N(R′)₂ groups wherein R is an alkyl, alkenyl, alkynyl or acyl group and R′ is, independent of other R′, selected from hydrogen, alkyl, alkyenyl, alkynyl or acyl, wherein one of aryl ring A or aryl ring B carry at least one non-hydrogen substituent and wherein the alkyl, alkenyl, alkynyl, acyl of R, R′ or R₁₋₃ are optionally substituted with one or more halogens, —CN, —NO₂, or —OH groups.

More specifically, the invention provides pyridine derivatives of formula II: 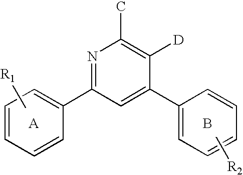
where:
A and B are aryl rings,
C is an aryl ring having an R₃ substituent and D is a hydrogen or a cyano group or C and D together form a 5- or 6-member optionally-substituted carbon ring in which one or two ring carbons are replaced with nitrogens;
R₁, R₂ and R₃ represent one or more than one substituent on the indicated ring, wherein each R₁, R₂ and R₃, independently of other R₁, R₂ and R₃ groups, are selected from hydrogen, —NO₂, —CN, halogen, alkyl, alkenyl, alkynyl, phenyl, —OH, —OR, —N(R′)₂ groups wherein R is an alkyl, alkenyl, alkynyl or acyl group and R′ is, independent of other R′, selected from hydrogen, alkyl, alkyenyl, alkynyl or acyl, wherein one of aryl ring A or aryl ring B carry at least one non-hydrogen substituent and wherein the alkyl, alkenyl, alkynyl, acyl of R, R′ or R₁₋₃ are optionally substituted with one or more halogens, —CN, —NO₂, or —OH groups.

In specific embodiments of the compounds of formula II:
one of aryl ring A or aryl ring B is substituted with an —OH group;
aryl ring A is substituted with an —OH group;
D is —CN;
C is an alkyl group, preferably a methyl group;
each of ring A and ring B carry at least one non-hydrogen substituent;
ring A carries a 4-O-alkyl group;
ring B carries a substituent other than —OMe or —N(CH₃)₂ in the 4-position;
ring B carries a substituent other than an alkoxyl or a dialkylamino group;
aryl ring a is an optionally-substituted phenyl ring;
aryl ring B is an optionally-substituted phenyl ring;
aryl ring A is a 4-OAlkyl substituted phenyl ring and aryl ring B is any aryl group other;
than an optionally substituted phenyl ring, e.g., aryl B ring is benzofuranyl;
aryl ring B is a halogen substituted phenyl ring;
C is an optionally substituted phenyl ring;

The invention provides pyridines of formula III: 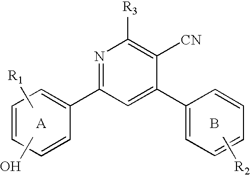
wherein R₃ is an alkyl or phenyl group, particularly a methyl group or a substituted phenyl group, where R₁ and R₂ are as defined in formulas I and II. In specific embodiments of formula III;
R₁ and R₂ are independently selected from hydrogen, —OH, halogen, alkyl or alkoxy;
the aryl B ring is benzofuranyl;
R₁ is hydrogen;
the aryl B ring is a phenyl ring; particularly a 4-N(CH₃)₂ substituted phenyl;
both of the aryl A and aryl B rings are phenyl rings.
the B ring is a halogen-substituted phenyl ring.

The invention provides pyridine derivatives of formula IV: 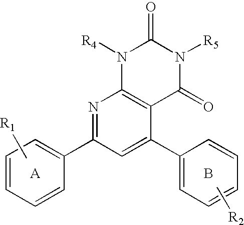
where:
R₄ and R₅, independently, are selected from alkyl groups having 1-6 carbon atoms and remaining variables are as defined above for formula I-III. In specific embodiments of formula IV:
R₁ and R₂ are independently selected from hydrogen, —OH, halogen, alkyl or alkoxy;
one of the aryl A ring or B ring is a phenyl ring;
the aryl B ring is benzofuranyl;
R₁ is hydrogen;
both of the aryl A and aryl B rings are phenyl rings;
the B ring is a halogen-substituted phenyl ring;
R₄ and R₅ are both methyl groups.

The invention provides compounds of formula V: 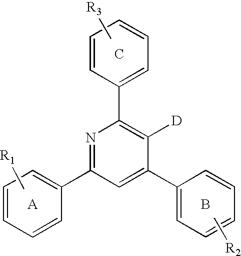
wherein the A, B and C rings are aryl rings;
D is hydrogen or a —CN group;
R₁, R₂ and R₃ represent one or more than one substituent on the indicated ring, wherein each R₁, R₂ and R₃, independently of other R₁, R₂ and R₃ groups, are selected from hydrogen, —NO₂, —CN, halogen, alkyl, alkenyl, alkynyl, aryl, arylalkyl, —OH, —OR, heterocyclic, heteroaryl, and —N(R′)2 groups wherein R is an alkyl, alkenyl, alkynyl, aryl, arylalkyl, heterocyclic, or heteroaryl group and R′ is, independent of other R′, selected from hydrogen, alkyl, aryl, arylalkyl, heterocyclic, or heteroaryl group wherein each of aryl ring A and aryl ring B carry at least one non-hydrogen substituent and wherein the alkyl, alkenyl, alkynyl, arylalkyl, heterocyclic, or heteroaryl groups of R, R′ or R1-3 are optionally substituted with one or more halogens, —CN, —NO₂, alkyl, alkenyl, alkynyl, or aryl groups. In specific embodiments of formula V:
D is H;
each of aryl rings A, B and C is different;
two of aryl rings A, B or C are the same;
the aryl rings A, B and C are all phenyl rings;
R₁ is not —OH or —NO₂;
R₂ is not —NO₂ or —CF₃;
R₁, R₂ and R₃ are not 4-chloro; 4-dimethylamine; 2-methyl, or 4-methoxy groups;
Aryl ring C is not a benzofuran or benzooxazole ring;
at least one of R₁, R₂ and R₃ is an alkyl or aryl amine group.

The invention provides compounds of formula VI: 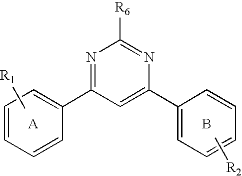
where
R₆ is selected from alkyl, aryl or —NRR″ where R and R″ are independently selected from hydrogen, alkyl, or aryl. R₁ and R₂ are as defined above for formulas I-V. In specific embodiments of formula VI:
R₆ is an alkyl having 1 to 6 carbon atoms;
R₆ is —NRR″;
R₆ is —NH2;
R₁ and R₂ are independently selected from hydrogen, —OH, halogen, alkyl or alkoxy.
the aryl B ring is benzofuranyl.
R₁ is hydrogen;
the aryl B ring is a phenyl ring.
both of the aryl A and aryl B rings are phenyl rings;
the B ring is a halogen-substituted phenyl ring.

The invention also provides methods for synthesis of triarylpyridines and particulary methods for synthesis of triarylpyridines on substrates. The synthetic method is applicable to the production of spatially addressable arrays of trialkylpyridines for used in screening for fluorescent molecules as described herein. Such synthetic methods and arrays are more generally useful for synthesis of arrays of increased chemical complexity which would be screened for any desired functional property. The methods herein are particularly useful for the synthesis of unsymmetric triarylpyridines.

More specifically, the invention provides a method for synthesizing a triarylpyridine which comprises the steps of: providing a surface-bound triaryl-1,5-dione; and condensing the surface-bound triaryl-1,5-diketone with NH₄OAc (ammonium acetate) to form a surface-bound triarylpyridine. The surface-bound triaryl-1,5-diketone can be formed by reaction of a surface-bound chalcone with a non-surface-bound aryl ketone. The surface-bound chalcone can be formed by reaction of a surface-bound aryl ketone with a non-surface-bound aryl aldehyde. In a specific embodiment, the surface-bound aryl ketone, the non-surface-bound aryl ketone and the non-surface-bound aryl aldehyde are different aryl rings, e.g., differently substituted aryl rings. The aryl ketones and the aryl aldehyde can each have at least one substituent on their aryl ring selected from —NO₂, —CN, halogen, alkyl, alkenyl, alkynyl, —OH, —OR, or —N(R′)₂ groups wherein R is an alkyl, alkenyl, alkynyl or acyl group and R′ is, independent of other R′, selected from hydrogen, alkyl, alkyenyl, alkynyl or acyl, and wherein the alkyl, alkenyl, alkynyl, or acyl of R and R′ are optionally substituted with one or more halogens, —CN, —NO₂, or —OH groups. The surface-bound aryl ketone, and the non-surface-bound aryl ketone can be acetophenones and the non-surface bound aryl aldehyde can be a benzaldehyde each of which can carry substituents on their aryl rings as noted. After synthesis on the array, one or more triarylpyridines can be removed from the array.

In other embodiments the triaryl-1,5-diketone can be formed by reaction of a surface-bound aryl aldehyde with a non-surface-bound aryl ketone. The surface-bound aryl aldehyde and the non-surface-bound aryl ketone can have the same or different substitution on their aryl rings which can be selected from groups as noted above. The surface-bound aryl aldehyde can be a benzaldehdye and the non-surface bound aryl ketone can be an acetophenone which can be substituted on their aryl rings as noted above. In a specific embodiment, the spatially-addressed array of triarylpyridines is formed on a unitary substrate.

The invention further includes improvements to SPOT-synthesis methods and particularly to MW-assisted SPOT-synthesis methods. Improvements include significant reductions in the time required to generate the linker-derivatized support.

The invention is further directed to macroarrays of compounds made by the methods herein and kits which comprise one or more compounds of this invention attached to a support, such as a planar support.

The invention is additionally directed to methods for using the compounds, identified by the methods herein, to be fluorescent or for which spectral properties were characterized herein in assays which employ fluorescent molecules. Fluorescent compounds of this invention are useful as research tools and in clinical applications and diagnostics, or example, to label biomolecules (i.e., peptides, proteins, and oligonucleotides), for use in various biological assays, both in vitro and in vivo. They are useful generally in any standard art-known assay which employs fluorescent molecules. They are further useful for the visualization of cellular processes and protein binding events, among many others. They are further useful for labeling DNA for gene chip analysis.

The invention also provides kits for detecting or labeling a biomolecule, kits for detection or measurement of pH, and kits for detection of a metal ion which comprised a planar support; and one more more fluorescent compounds of the above formulas bound to the support. A kit can be a kit for detecting or labeling a nucleic acid, peptide, protein, or an antibody. The kit for detecting or labeling a biomolecule can be a kit useful for labeling cellular compartment or organelles.

Other objects and advantages are apparent from the drawings, description of the drawings, the detailed description of the invention and the appended claims.

### BRIEF DESCRIPTION OF THE DRAWINGS

FIG. 1 illustrates a schematic for SPOT synthesis indicating the use of microwave to accelerate synthesis.

FIG. 2 illustrates a sub-section of a cyanopyridine macroarray irradiated at 254 nm using a handheld UV lamp, showing three rows differing by the nature of the R₁ substituent (4-OH, 3-OH and 3-OMe, 4-OH) and five columns differing by the nature of the R₂ substituent (4-NMe₂, H, 2-F and 3-F, 4-F).

FIG. 3 shows plots of the emission spectra of cyanopyridine 2h irradiated at 340 nm in various solvents: tetrahydrofuran (THF), chloroform (trichloromethane, CHCl3), dimethyl sulfoxide (DMSO) and ethanol (EtOH).

FIG. 4 is a graph of the excitation and emission spectra of purified cyanopyridine 2h obtained from solution-phase synthesis.

FIG. 5 is a graph of the excitation and emission spectra of purified cyanopyridine 2g obtained from solution-phase synthesis.

FIG. 6 is a graph of the excitation and emission spectra of purified deazalumazine 3c obtained from solution-phase synthesis.

### DESCRIPTION OF THE PREFERRED EMBODIMENTS

In this invention small molecule fluorophores are constructed and screened directly on an array. Schena, M.; Shalon, D.; Davis, R. W.; Brown, P. O. Science 1995, 270, 467-470. Arrays of surface-bound candidates are screened to identify compounds with desirable fluorescent properties. Array screens have been used herein to identify heterocyclic fluorophores exhibiting useful fluorescent properties. The screens have been used herein to identify certain cyanopyridine, deazalumazine, triarylpyridine and pyrimidine structures which exhibit useful fluorescent properties. The array screening methods herein can also be used to compare structural variants of candidate fluorescent cores to evaluate the effect of variation in structure, such as variation in ring substitution, on fluorescent properties. The information obtained from screening can be used to identify new fluorescent cores and molecules and, at least in part, characterize fluorescence from such new cores and molecules. The information obtained by array screening of a set of structurally related fluorophores can for example be employed to facilitate selection of an appropriate fluorophore for use in a given application. The information obtained can also be used to guide synthesis of new fluorescent cores and molecules which are then prepared by conventional synthetic methods and characterized by conventional spectroscopic methods. The combined array synthesis and screening methods herein can provide rapid assessment of fluorescence from a plurality of structurally related fluorophores to facilitate application of those fluorophores and the development of additional useful fluorophores. A portion of the work described herein has been reported in Bowman, M. D., Jacobson, M. M, and Blackwell, H. E. Org. Lett. 2006, 8:1645-1648; Bowman, M. D., Jacobson, M. M, Pujanauski, B. G. and Blackwell, H. E. Tetrahedron (Symposium-in-Print on Microwaves in Organic Chemistry) 2006, 62, 4715-4727; and Bowman, M. D., Jeske, R. C., and Blackwell, H. E. Org. Lett. 2004, 6:2019-2022.

The present invention generally provides libraries of heterocyclic fluorophores in array format, and particularly in macroarray format, constructed via efficient spatially addressed synthesis on unitary supports or substrate. A unitary support consists of a single support element containing all members of a given array in contrast to an array comprises of a plurality (typically one for each array member) of substrates, such as arrays on a plurality of beads. Construction of the array on a planar substrate facilitates screening of fluorescence. The methods of this invention can be practiced employing any array format including microarrays and macroarrays. Macroarrays are the preferred format. The quantity of product generated on a macroarray (˜100 nmol) is sufficient to provide for a full spectral characterization of the library compounds.

Employing screens of this invention, as well as information derived from such screens, a set of exemplary compounds with useful fluorescent properties have been identified. For example, compounds with promising solvatofluorochromic properties, such as cyanopyridine 2h and exceptionally high quantum yields, such as cyanopyridines 2r and 2t have been identified. These results demonstrate the value of small molecule macroarrays for fluorescent dye discovery and development.

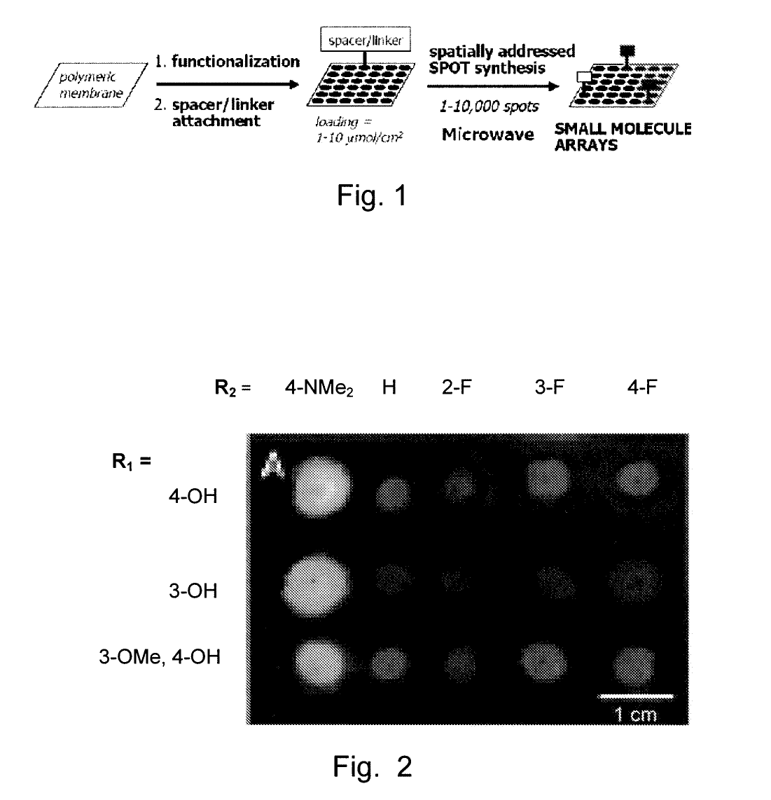

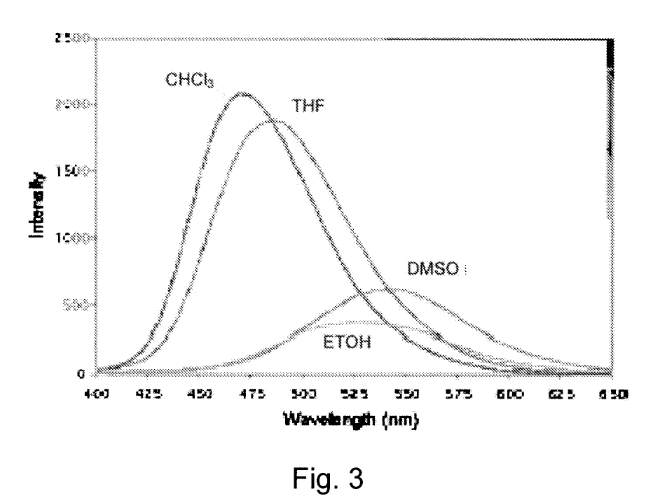

Macroarrays of candidate fluorescent molecule are constructed using the SPOT-synthesis technique (Frank, R. Tetrahedron 1992, 48, 9217-9232; Frank, R. J. Immunol. Methods 2002, 267, 13-26) which is schematically illustrated in FIG. 1. As indicated, microwave-accelerated SPOT synthesis can be employed. As will be discussed below, conventional heating can also be used in array synthesis and, in some cases, improvements in conversion are obtained as compared to use of microwave radiation. SPOT-synthesis is a robust method for generating spatially-addressed libraries (an array) (Bowman, M. D.; Jeske, R. C.; Blackwell, H. E. Org. Lett. 2004, 6, 2019-2022). In this approach, small molecules are synthesized directly on the planar support; unlike small molecule microarray construction, where compounds are generally attached to the microarray post-synthesis (Uttamchandani, M.; Walsh, D. P.; Yao, S. Q.; Chang, Y. T. Curr. Opin. Chem. Biol. 2005, 9, 4-13.) Cellulose paper has been used historically for SPOT-synthesis, as it is derivatized easily, straightforward to manipulate, and inexpensive. Whatman 1Chr chromatography paper was used for SPOT-synthesis (thickness=0.34 mm and cost ˜0.5¢/cm²) herein.

The synthesis of a chalcone library using this technique, as illustrated in Scheme 1, has been reported (Bowman, M. D.; Jeske, R. C.; Blackwell, H. E. Org. Lett. 2004, 6, 2019-2022). In this scheme, a set of aryl ketones 50 with R₁ substituent and a hydroxyl for attachment to the surface is initially linked to the substrate. In Scheme 1, attachment is illustrated as via a 4-OH, but the hydroxyl for attachment can be at any appropriate position on the aryl ring. A set of aryl aldehydes 51 carrying an R₂ substituent is reacted with the surface-bound ketone as indicated to form the diaryl chalcone. The aryl ketones provide the aryl A ring of later schemes and the aryl aldehydes provide the aryl B ring of later schemes. 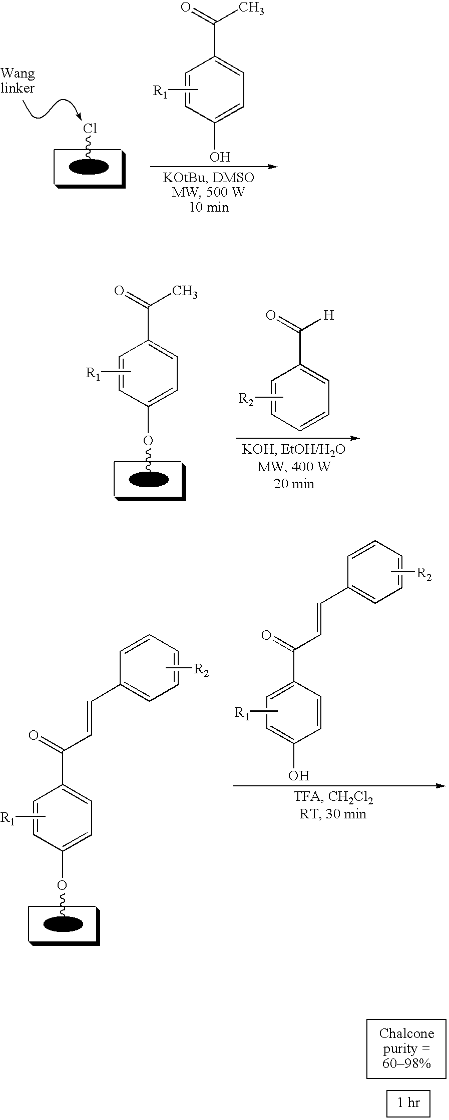
Scheme 1: Preparation of chalcone (1) library.

In the chalcone library, the inventors introduced a cellulose support system derivatized with an amine spacer and an acid-cleavable, Wang-type linker. Bowman, M. D.; Jeske, R. C.; Blackwell, H. E. Org. Lett. 2004, 6, 2019-2022. A general schematic illustrating use of linkers is shown in Scheme 2.

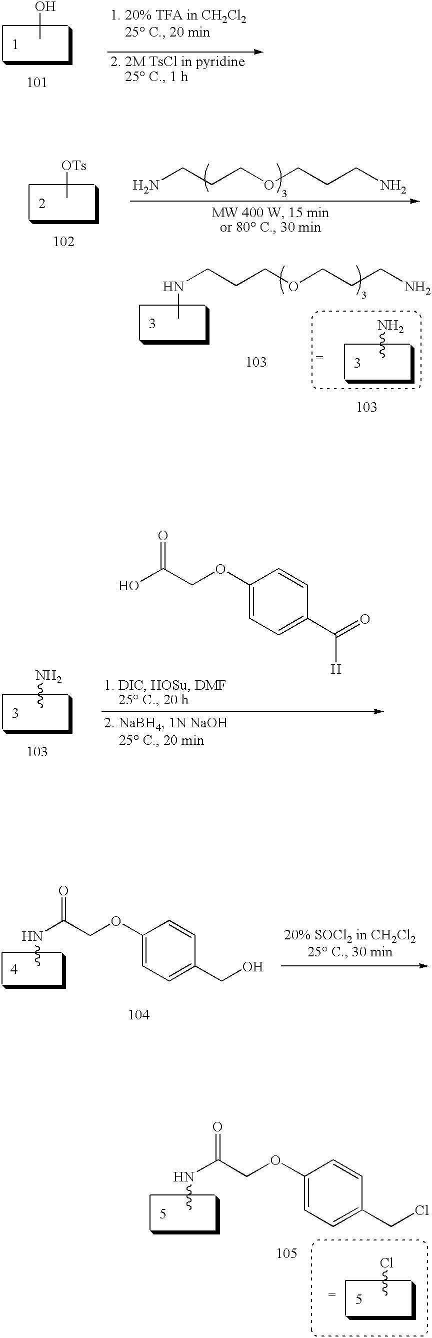

Scheme 2. Preparation of Wang-linker derivatized support 104 and chloride-derivatized, “activated linker”, support 105. Supports were washed with various solvents and dried routinely between each synthetic step.

Second generation libraries can be formed by subsequent reaction of the alpha-beta unsaturated chalcone and various reactants, reagents or both. Of particular interest for this invention are reactions in which a heterocyclic ring is formed on reaction with the surface-bound chalcone. Powers, D. G. et al. Tetrahedron 54 (1998):4085-4096 and International Patent Application WO 98/15532 (Martini) provide details of a number of reactions of alpha-beta unsaturated ketones useful for synthesis of libraries of this invention.

Schemes 3-11 provide exemplary reactions useful for synthesis of candidate fluorescent molecule arrays of this invention.

Scheme 3: Cyanopyridine (2) library synthesis:

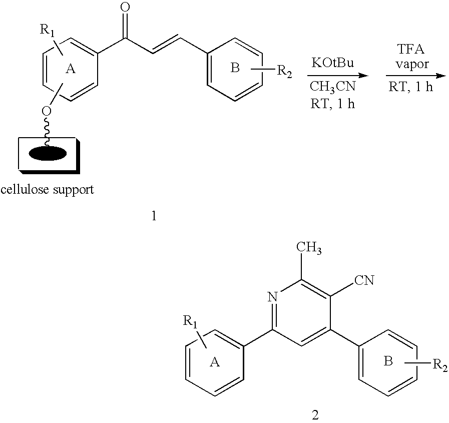

Additional cyanopyridines can be prepared by replacing the methyl group of the pyridine ring with R₈, where R₈ is an alkyl, phenyl or aryl group which may carry an R₃ substituent as defined below. Reactants and/or reagents are readily selected to prepare such derivatives. In specific embodiments, the aryl B ring is benzofuran.

Scheme 4: Deazalumazine (3) library synthesis:

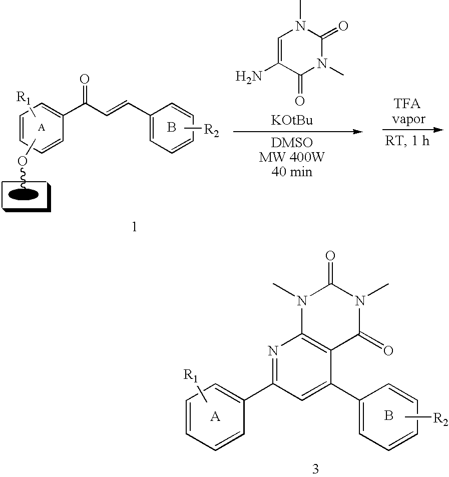

Additional deazalumazines can be prepared by replacing the one or two of the methyl group of the pyridine ring with R₈, where R₈ is independent of other R₈, an alkyl, phenyl or aryl group which may carry an R₃ substituent as defined below. Reactants and/or reagents are readily selected to prepare such derivatives. In specific embodiments, the aryl B ring is benzofuran.

Scheme 5: Pyrimidine (4) library synthesis:

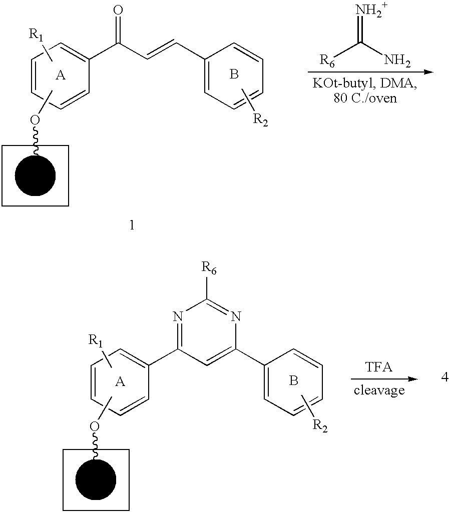

In Scheme 5, R₆ is selected from alkyl, aryl, phenyl or —NRR″ where R and R″ are independently selected from hydrogen, alkyl, aryl or phenyl. In specific embodiments, the aryl B ring is benzofuran.

Scheme 6: Pyridine derivatives (25) library synthesis:

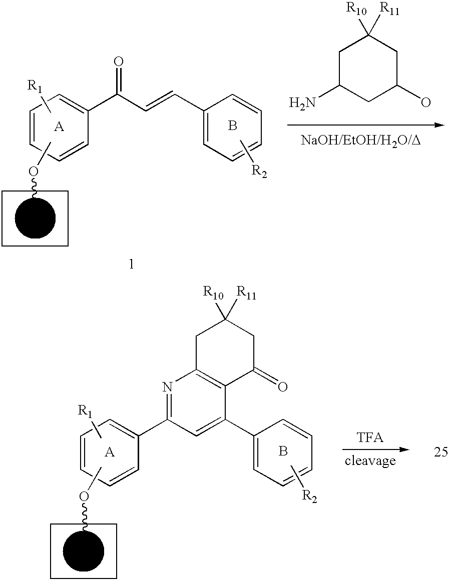

In Scheme 6, R₁₀ and R₁₁ can be the same or different and are independently selected from alkyl, aryl or phenyl groups.

Scheme 7: Isoxazoline derivative (30) library synthesis:

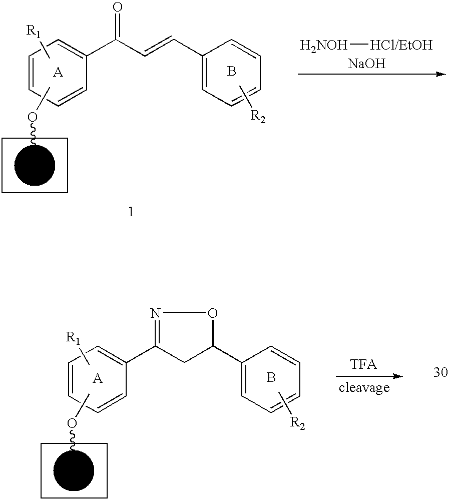

Scheme 8: Diaryl-N-arylpyrazoline (35) library synthesis:

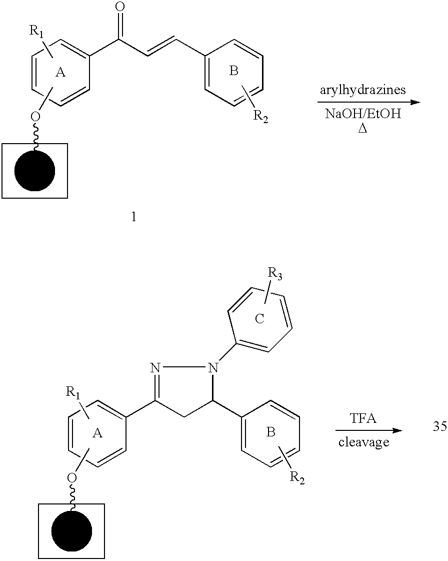

Scheme 9: Diarylpyrazole (40) library synthesis:

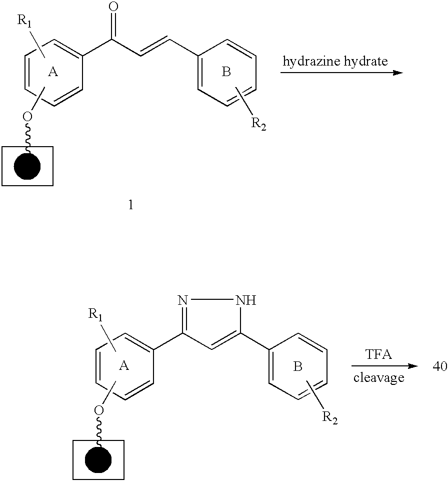

Scheme 10: Diaryl tetrahydropyrimidines (45) library synthesis:

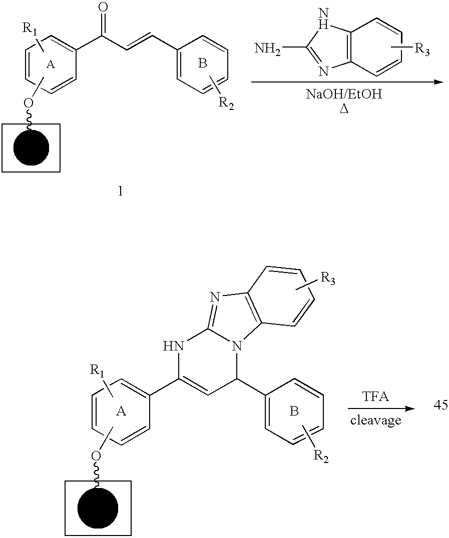

Scheme 11: Synthesis of a library of Spiro-polyheterocyclic isatin-chalcone adducts (49)

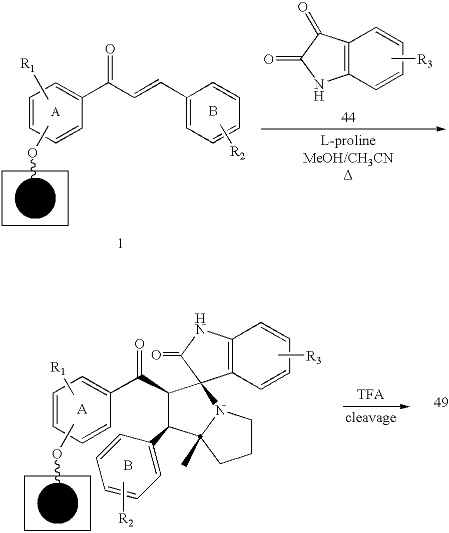

In Scheme 11, isatin 44 is combined with an amino acid, L-proline is illustrated in the scheme, to form the complex heterocycle 49. As is known in the art this reaction can combined different amino acids which lead to the formation of different adduct structures.

In all of the above schemes the aryl A, aryl B and aryl C rings can be any aryl ring, R₁, R₂ and R₃, can represent one or more than one substituent on the indicated ring, wherein each R₁, R₂ and R₃, independently of other R₁, R₂ and R₃ groups, are selected from hydrogen, —NO₂, —CN, halogen, alkyl, alkenyl, alkynyl, aryl, phenyl, —OH, —OR, —N(R′)₂ groups wherein R is an alkyl, alkenyl, alkynyl, aryl, phenyl or acyl group and R′ is, independent of other R′, selected from hydrogen, alkyl, alkyenyl, alkynyl, aryl, phenyl or acyl, and wherein the alkyl, alkenyl, alkynyl, acyl, aryl or phenyl of R, R′ or R₁₋₃ are optionally substituted with one or more halogens, —CN, —NO₂, or —OH groups. and other variable are as defined.

Preferred substituents for the ketones and aldehydes of Scheme 1 are those that do not interfere with the aldol condensation and which do not lead to significant side reactions which decrease conversion to chalcones. Preferably, in the benzaldehyde R₂ is not —NO₂ or —CF₃. Benzaldehydes bearing —NO₂ and —CF₃ groups have been found to be too active in the aldol reaction (Scheme 1) leading to a complex mixture of products. Similarly, —NO₂ groups are not preferred on the acetophenone because they tend to make the ring too active and multiple side reactions leading to complex mixtures of products are obtained in the aldol condensation. Also preferably, in the acetophenone only one —OH is present, which is used to attach the acetophenone to the support. The presence of additional OH groups suppress chalcone formation through quenching of the basic catalyst. Additional OH groups could be accommodated through the use of protecting groups (e.g., alkoxides), which could be removed after the aldol reaction is completed. However, the use of such protecting groups can add significant complexity to the reactions.

In the present invention, the chalcone scaffold is readily transformed into a variety of heterocycles some of which are exemplified in the above schemes. To illustrate the methods herein chalcone arrays were converted into exemplary nitrogen-containing heterocycles to examine their fluorescent properties. Two structural classes were selected for initial investigations, cyanopyridines and deazalumazines, which both can be generated in one-step from chalcones and have yet to be characterized broadly as dye molecules. Matsui, M.; Oji, A.; Hiramatsu, K.; Shibata, K.; Muramatsu, H. J. Chem. Soc. Perkin Trans. 2 1992, 2, 201-206. Initial libraries were generated from a 36-membered chalcone array 1 (Scheme 1), synthesized as previously reported (Bowman, et al., 2004). The chalcone array was formed using three different acetophenones (4′-OH acetophenone, 3′-hydroxyacetophenone and acetovanillone (R₁=3′-OMe, 4′-OH) and 12 different benzaldehydes (R₂=H, 2-F, 3-F, 4-F, 3-Br, 4-Br, 4-Cl, 3,4-difluoro, 3-OMe, 4-OMe, and 4-dimethylamino). Additional details are reported in Bowman et al. 2006, Org. Lett. 8:1645-1648.)

In a subsequent array synthesis, the initial chalcone library was expanded by employing an additional benzaldehdye derivative, piperonal (3,4-(methylenedioxy)-benzaldehyde which was reacted with the three listed acetophenones to give an array of chalcones in which the aryl B ring is benzofuran. Cyanopyridines and deazalumazines were prepared from this chalcone library having a benzofuran B-ring. The chalcone library can be further expanded by employing 2,3-(methylenedioxy)-benzaldehyde, 4′-formylbenzo-15-crown-5,6-nitropiperonal, 3,4-dibenzyloxybenzaldehyde and 2,3-dibenzyloxybenzaldehyde. The Aryl B ring can thus be expanded to heterocylic rings and the R₂ groups can be expanded to additional aryl oxy groups and to crown ethers.

The route to cyanopyridines (2) from the chalcone scaffold (1) employs in situ generation of 2-aminocrotylnitrile from acetonitrile through sonication in the presence of potassium tert-butoxide (Scheme 3). Marzinzik, A. L.; Felder, E. R. J. Org. Chem. 1998, 63, 723-727. The resulting mixture was “spotted” onto the support four times at room temperature at ten minute intervals to generate cyanopyridine array 2. A range of functionality incorporated in the original A and B rings of the chalcone were tolerated by this reaction.

After washing and drying of the membrane, the spectral differences can be observed directly on the support by simply irradiating the paper with a handheld UV-lamp (FIG. 2). In this way, structure-activity relationships across the entire array can be qualitatively derived. For example, in FIG. 2 it is possible to elucidate several important structure-activity relationships. Molecules across each row have the same A ring substitution. The middle row is dimmer, having meta hydroxyl groups on the A ring. The top and bottom rows have hydroxyl groups at the para position. Compounds in each column have the same B ring. The dramatic effect of incorporation of a dimethylamino group in the B ring is apparent in the far-left column. The simplicity of and ease of obtaining information from this initial screen for fluorescence properties highlights other advantages of the use of SPOT-synthesis to generate these arrays for fluorescent screening.

Table 1 provides structures and spectral properties of selected cyanopyridine library members. Additionally, Table 1 includes data for several cyanopyridines synthesized in solution. For quantitative analysis, the cyanopyridines were cleaved from the support using trifluoroacetic acid (TFA). Sufficient quantities of each compound (ca. 100 nmol) were obtained from a single spot to determine excitation and emission spectra as well as quantum yields. In fact, to obtain a sample sufficiently dilute for the determination of quantum yield, the amount obtained from one spot had to be dissolved in 20 mL of solvent. Product purity was determined by LC-MS analysis of a random sample (30%) of compounds, all of which were between 70 and 90% pure, as determined by integration of the HPLC trace at 254 nm.

Quantitatively derived structure-activity relationships largely agreed with those observed directly from the array. All of the fluoresecent cyanopyridines have large Stokes shifts (90-120 nm). For comparison, fluorescein, one of the most commonly used fluorophores, has a Stokes shift of 19 nm (Urano, Y., et al., J. Am. Chem. Soc. 2005, 127, 4888-4894). Large Stokes shifts are a highly desirable characteristic for practical applications of fluorescent dyes due to the minimization of reabsorption of the emitted light by the solution (Jameson, D. M., et al., Methods Enzymol. 2003, 360, 1-43).

The results of these assays indicate that a hydroxyl or other electron donating group in the para position of the A ring greatly enhances fluorescence (2a-2p), while cyanopyridines that bear only a meta hydroxyl group (2q) have very low quantum yields (<0.01). Additional incorporation of electron donating groups to the A ring produces higher wavelength emission, but reduced quantum yields.

Alterations to the substituents of the B ring, with one notable exception, had little effect on the emission spectra of these cyanopyridines. Due to their strong electron donating capability, dialkylamino groups are often incorporated into environmentally sensitive probes (Weber, G.; Farris, F. J. Biochemistry 1979, 18, 3075-3078). Inclusion of a para dimethylamino substituent into the cyanopyridines had a profound effect on the spectral characteristics. Fluorescence spectra of 2h and 2p display significant red shifts (91 nm and 64 nm respectively) compared to the other cyanopyridines studied. Visually, while all of the other cyanopyridines fluoresce blue, ethanolic solutions of the dimethylamino-substituted compounds appear yellow-green.

For further analysis, 2g and 2h were synthesized in solution. Their spectral characteristics matched those of the compounds from the macroarray. The evaluation of pH dependence was studied to ensure that residual TFA from the cleavage step would not alter results. Buffered ethanolic solutions of 2g and 2h were studied over a wide pH range. The excitation and emission wavelengths were unchanged in pH ranges from 1.6 to 10. While the quantum yield of 2g was nearly constant from low to neutral pH, the quantum yield of 2h varied. The quantum yield was 0.06 at pH 1.6, while that at neutral pH was 0.09. The quantum yield of the sample from the array matched that of the solution-phase at pH 7.6 confirming that the products are the neutral compounds rather than TFA adducts and that little residual TFA is present to alter results. At pH >7.6, the quantum yields of both 2g and 2h began to diminish.

Under the assumption that the quantum yield was being decreased due to deprotonation of the phenol, the hydroxyl group of several of the cyanopyridines was replaced with a methyloxy group in a solution-phase synthesis to increase the independence of the dyes from high pH. Spectral properties of these cyanopyridines are included in Table 1.

Additionally, it had been observed that before cleavage from the substrate that the brightest Spots in the array were consistently those derived from acetovanillone (2i-2p). Interestingly, after cleavage and elution, analysis showed that these were not, in fact, the compounds with the highest quantum yield. Again, it was hypothesized that alkylation of the hydroxyl group would prove beneficial. Methylated versions of 2b, 2g, and 2o were synthesized resulting in 2r, 2s, and 2t, respectively. The quantum yields of these methylated dyes were increased by five to tenfold over their parent cyanopyridines, placing these fluorophores in the range of common coumarin (Besson, T, et al., J. Heterocycl. Chem. 1991, 75, 1517-1523) and fluorescein (Urano, et al., 2005) derivatives. As anticipated, the spectral properties of 2r-2t were unaffected by pH.

Numerous cyanopyridines are reported in the literature: Muramatsu, Hiroshige; Shibata, Katsuki; Matsui, Masaki. “Preparation of diphenylcyanopyridine derivatives as fluorescent dyes,” Jpn. Kokai Tokkyo Koho (1992 JP 04279568 A2 19921005 Heisei; Marzinzik, Andreas; Felder, Eduard. “Solid phase synthesis of heterocyclic compounds,” PCT Int. Appl. (1998) WO 9815532 A1; Matsui, Masaki; Oji, Akira; Hiramatsu, Koichi; Shibata, Katsuyoshi; Muramatsu, Hiroshige, “Synthesis and characterization of fluorescent 4,6-disubstituted-3-cyano-2-methylpyridines,” Journal of the Chemical Society, Perkin Transactions 2 (1992), (2), 201-6; Marzinzik, Andreas L.; Felder, Eduard R., “Key Intermediates in Combinatorial Chemistry: Access to Various Heterocycles from a,b-Unsaturated Ketones on Solid Phase,” Journal of Organic Chemistry (1998), 63(3), 723-727.

For the array synthesis of deazalumazines (Scheme 4), the benzaldehyde R₂ group is preferably not —OH, —NO₂, —CF₃, or —NRR′. When R₂ is —OH, the basic catalyst is quenched by the presence of the acidic phenol and the resulting substrate is deactivated toward nucleophilic attack. When R₂ is —NRR′, the chalcone is deactivated by the increased electron density in the α,β-unsaturated carbonyl system by the strongly donating amino substituents. Benzaldehydes bearing —NO₂ and —CF₃ groups are too active in the adol reaction leading to a complex mixture of products. Preferably, the acetophenone has only one OH, which is that used to attach the acetophenone to the support. Additional —OH groups suppress chalcone and deazalumazine formation through quenching the basic catalyst. NO₂ groups on the acetophenone ring tend to make the ring too active and multiple side reactions leading to complex mixtures of products are obtained in the aldol condensation. As noted above, the use of protecting groups, might facilitate or improve synthesis with certain R₁ and R₂ groups, but would result in more complex synthesis.

For the deazalumizine library, an array of deazalumazines from the same chalcone basis set used for the cyanopyridines was prepared. The spotting solution consisted of an aminouracil and hydroxide. As in the cyanopyridine synthesis, the spotted membrane to stand at room temperature for ten minutes. However, this gave only 10-15% conversion even after spotting four times. By spotting and microwaving the membrane four times in succession, complete conversions were obtained with purities of most compounds ranging from 70-90%. The para dimethylamino chalcones 1h and 1p, which displayed such interesting changes in properties in the cyanopyridine array, were not converted to the target deazalumazines under these conditions. It is believed that the increased electron-donation into the π-system in these cases is inhibiting nucleophilic attack on the α,β-unsaturated system.

TABLE 1
Structures and spectral properties of cyanopyridine library (2).

| entry | R¹ | R² | λₑₓ | λₑₘ | φ_{f}^{a} |
|---|---|---|---|---|---|
| 2a | 4-OH | H | 341 | 433 | 0.07 |
| 2b | 4-OH | 2-F | 342 | 430 | 0.06 |
| 2c | 4-OH | 4-F | 341 | 430 | 0.07 |
| 2d | 4-OH | 3-Br | 344 | 445 | 0.03 |
| 2g | 4-OH | 4-OMe | 339 | 423 | 0.12 |
| 2h | 4-OH | 4-NMe₂ | 366 | 524 | 1.09 |
| 2i | 4-OH, 3-OMe | H | 351 | 469 | 0.03 |
| 2j | 4-OH, 3-OMe | 2-F | 351 | 465 | 0.02 |
| 2k | 4-OH, 3-OMe | 4-F | 351 | 466 | 0.04 |
| 2l | 4-OH, 3-OMe | 3-Br | 353 | 477 | 0.02 |
| 2o | 4-OH, 3-OMe | 4-OMe | 351 | 461 | 0.06 |
| 2p | 4-OH, 3-OMe | 4-NMe₂ | 348 | 533 | 0.09 |
| 2q | 3-OH | 4-OMe | 326 | 420 | <0.01 |
| 2r^{b} | 4-OMe | 2-F | 336 | 419 | 0.71 |
| 2s^{b} | 4-OMe | 4-OMe | 335 | 409 | 0.77 |
| 2t^{b} | 4-OMe, 3-OMe | 4-OMe | 344 | 448 | 0.74 |

^{a}Data recorded from crude samples. Quantum yields measured in ethanol. External standard: coumarin 120 (φ_{f} = 0.88, λ_{exc} = 354 nm, λₑₘₘ = 435 nm in ethanol). Error = ±15%.
^{b}Synthesized in solution.

Table 2 reports spectral properties of selected deazalumizines of formula 3.

TABLE 2

| entry | R¹ | R | λ(nm) | λ(nm) | φ_{f} |
|---|---|---|---|---|---|
| 3a | 4-OH | H | 362 | 446 | 0.15 |
| 3b | 4-OH | 2-F | 363 | 443 | 0.18 |
| 3c | 4-OH | 4-F | 364 | 445 | 0.19 |
| 3d | 4-OH | 3-Br | 364 | 452 | 0.07 |
| 3g | 4-OH | 4-OMe | 365 | 438 | 0.03 |
| 3i | 4-OH, 3-OMe | H | 368 | 480 | 0.06 |
| 3j | 4-OH, 3-OMe | 2-F | 371 | 485 | 0.06 |
| 3k | 4-OH, 3-OMe | 4-F | 370 | 483 | 0.07 |
| 3l | 4-OH, 3-OMe | 3-Br | 370 | 489 | 0.03 |
| 3o | 4-OH, 3-OMe | 4-OMe | 372 | 474 | 0.06 |
| 3u | 3-OH | 4-F | 367 | 441 | 0.05 |
| 3v | 3-OH | 4-Cl | 356 | 440 | <0.01 |
| 3w | 4-OMe | 4-F | 360 | 424 | 0.19 |

Data recorded from crude samples. Quantum yields measured in ethanol. External standard: coumarin 120 (φ_{f} = 0.88, λ= 354 nm, λ= 435 nm in ethanol). Error = ±15%. Synthesized in solution.

Quantum yields of the deazalumazines were generally markedly higher than those of the cyanopyridines. As in the cyanopyridine cases, substitution of the A ring had the most influence over the spectral properties in terms of excitation/emission wavelengths and quantum yield. B ring alterations only impacted the quantum yield. Due to its high quantum yield, deazalumazine 3c ws synthesized in solution for further analysis. The spectral characteristics of 3c were within experimental error of those of the crude sample cleaved from the macroarray. Again similar to the cyanopyridines, pH had no effect on the emission wavelength and the quantum yield began to diminish in basic solutions. By substituting a methoxy group for the hydroxyl on 3c, deazalumazine 3w was prepared. Although 3w has a smaller Stokes shift than its parent 3c (64 nm vs. 81 nm), the fluorescence of 3w is completely pH-independent with a quantum yield of 0.19. However, the significant quantum yield increase seen in the cyanopyridines was not seen in the deazalumazines when the phenol was alkylated.

Cyanopyridines and deazalumazines in which the aryl B ring is benzofuranyl exhibited quantum yields in the range of 0.20-0.40, with Stokes shifts in the range of 100 nm. The affect of variation of A ring substitution on spectral properties of the cyanopyridines and deazalumazines where the B ring is benzofuranyl were similar to those observed for other cyanopyridines or deazalumazines.

Compounds having structures related to deazalumizines are discussed in the literature: Tominaga, Takeshi; Murase, Seiichiro; Kohama, Toru, “Red-emitting organic electroluminescent devices with high electric energy conversion efficiency and color purity,” Jpn. Kokai Tokkyo Koho (2002) JP 2002008862 A2; Levy, Stuart B.; Alekshun, Michael N.; Podlogar, Brent L.; Ohemeng, Kwasi; Verma, Atul K.; Warchol, Tadeusz; Bhatia, Beena, “Transcription factor modulating compounds and methods of use thereof,” U.S. Pat. Appl. Publ. (2003), 301 US 2003229065 A1; Levy, Stuart B.; Alekshun, Michael N.; Podlogar, Brent L.; Ohemeng, Kwasi; Verma, Atul K.; Warchol, Tadeusz; Bhatia, Beena; Bowser, Todd; Grier, Mark, “Substituted benzoimidazole compounds as transcription factor-modulating compounds useful as anti-infectives,” U.S. Pat. Appl. Publ. (2005) US 20050124678 A1; Bosse, Dieter; Wingen, Rainer; Horn, Klaus; Lutz, Walter, “10-Phenyl-1,3,9-triazaanthracenes and photopolymerizable mixture containing them,” Ger. Offen. (1984) DE 3232620 A1 19840308; Bailey, Joseph; Elvidge, John A, “Synthesis and properties of dyes containing the pyrano[2,3-d]pyrimidine nucleus.” Journal of the Chemical Society, Perkin Transactions 1: Organic and Bio-Organic Chemistry (1972-1999) (1973), No. 8 823-8.

The environmental sensitivity of a fluorophore is can be crucial to its application. Solution-phase standards 2g, 2h and 3c were examined in a variety of solvents to determine the effects of solvent polarity (Table 3). The deazalumazine emission spectra underwent a significant bathochromic shift in fluorescence wavelength as well as increased quantum yield with increased solvent polarity. The cyanopyridines display a similar trend in spectral shift with changing solvent polarity; however, the quantum yield trend is reversed and more pronounced. For example, the quantum yield of 2g is quadrupled in THF over that in ethanol.

TABLE 3
Influence of solvent on dye spectral properties.

| entry | solvent | λ (nm) | λ. (nm) | φ |
|---|---|---|---|---|
| 2g | THF | 339 | 405 | 0.66 |
| 2g | CHCl | 333 | 404 | 0.58 |
| 2g | DMSO | 344 | 440 | 0.31 |
| 2g | EtOH | 341 | 424 | 0.15 |
| 2h | THF | 373 | 486 | 0.53 |
| 2h | CHCl | 372 | 471 | 0.53 |
| 2h | DMSO | 379 | 538 | 0.17 |
| 2h | EtOH | 379 | 526 | 0.10 |
| 3c | THF | 361 | 417 | 0.12 |
| 3c | CHCl | 361 | 417 | 0.13 |
| 3c | DMSO | 363 | 454 | 0.23 |
| 3c | EtOH | 363 | 442 | 0.23 |

External standard: coumann 120 (φ = 0.88, λ = 354 nm, λ. = 435 nm in ethanol).
Error = ±15%.

Dimethylamino substituted cyanopyridine 2h proved to be the most solvent sensitive probe of the fluorophores studied. The spectral differences are visually observable (see spectral in FIG. 2) with polarity differences as small as those between THF and CHCl₃ evident. The methoxy substituted analog (2g) was not able to make this distinction. Such large emission changes, with little dependence on pH, make 2h an attractive candidate to examine polarity changes at cellular membranes (Cohen, B. E., et al., Proc. Natl. Acad. Sci. U.S.A. 2005, 102, 965-970).

Array libraries generated by the reactions of Schemes 5-11 will provide useful fluorescent molecules.

Synthesis of Triarylpyridine Arrays

The use of hydroxybenzaldehydes, instead of hydroxyactophenones as the support-bound reactant in the Claisen-Schmidt condensation was investigated as an approach to increase the structural complexity of arrays that could be generated using the methods herein. A substantially larger number of substituted hydroxybenzaldehyde building blocks are commercially available compared to substituted hydroxyacetophenones. It was found that the loading conditions used to attach hydroxyacetophenones to the support could also be used to load hydroxybenzaldehydes to the support with analogous compound loadings (ca. 290 nmol/cm²). However, during the subsequent Claisen-Schmidt condensation step between support-bound hydroxybenzaldehydes and solution-phase acetophenones, two competing reaction pathways were observed. A representative reaction of support-bound vanillin (10) with 3′-methoxyacetophenone (11) is:

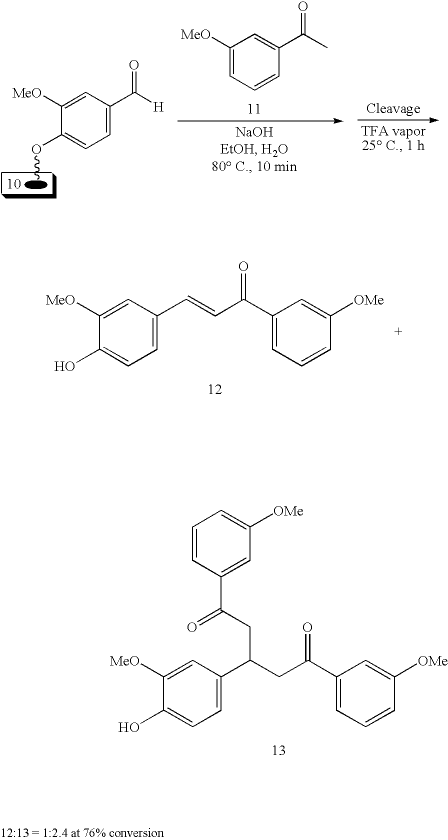

The desired Claisen-Schmidt condensation reaction between 10 and 11 was found to compete with Michael addition of 11 to the newly formed chalcone. Even after one application of the acetophenone (11) solution, three distinct species could be observed after 10 min at 80° C. (in the oven): (1) the starting material vanillin, (2) the target chalcone 12, and (3) a byproduct 13 (as determined post-cleavage). The latter compound was the major product (2.4:1 13:12 at 76% conversion). Its identity as the 1,5-diketone 13 was confirmed by comparison to an authentic sample of 13 that was synthesized and characterized separately.

The side reaction to generate diketone 13, while initially unwanted, proved fortuitous. By spotting vanillin-derived support 10 with acetophenone 11 and heating four times in succession (80° C. in the oven, 10 min each), the reaction was driven predominantly to the 1,5-diketone product 13 (88% conversion). This new reaction pathway is useful for the construction of additional small molecule macroarrays with increased structural complexity.

The surface-bound array of 1,5-diketones (13) in general represents a new starting material of synthesis of macroarrays of increasing complexity and is more specifically useful in the methods of this invention for synthesis of candidate fluorescent molecules for screening.

Diketones such as 13 have been reported as precursors to 2,4,6-triarylpyridines (Kröhnke, F. Synthesis 1976, 1-24; Cave, G. W. V.; Raston, C. L. Chem. Commun. 2000, 22, 2199-2200). Triarylpyridines have found wide use as building blocks for supramolecular chemistry (Constable, E. C.; Harverson, P.; Smith, D. R.; Whall, L. A. Tetrahedron 1994, 50, 7799-7806; Zimmerman, S. C.; VanZyl, C. M.; Hamilton, G. S. J. Am. Chem. Soc. 1989, 111, 1373-1381) and as chemosensors (Mello, J. V.; Finney, N. S. J. Am. Chem. Soc. 2005, 127,10124-10125; Fang, A. G.; Mello, J. V.; Finney, N. S. Tetrahedron 2004, 60, 11075-11087) and the development of efficient methods to generate this class of compounds has attracted considerable interest. Traditional syntheses of triarylpyridines involving acetophenones and benzaldehydes are low yielding, due to the intermediate dihydropyridine reducing the intermediate chalcone (Weiss, M. J. Am. Chem. Soc. 1952, 74, 200-202.) To alleviate this unwanted side reaction, Kröhnke developed an elegant alternative utilizing pyridinium salts (Kröhnke, F. Synthesis 1976, 1-24.) Triarylpyridines are also useful as chemosensors for cations, as described in J. V. Mello & N. S. Finney Angew. Chem. Int. Ed. 2001, 40, 1536.and for numerous other applications.

MW-assisted and solid-phase synthetic routes to triarylpyridines from chalcone precursors have been reported (Tu, S.; Li, T.; Shi, F.; Fang, F.; Zhu, S.; Wei, X.; Zong, Z. Chem. Left. 2005, 34, 732-733; Grosche, P.; Höltzel, A.; Walk, T. B.; Trautwein, A. W.; Jung, G. Synthesis 1999, 11, 1961-1970.) This aspect of the invention relates to MW-assisted synthesis of triarylpyridines from support-bound diketones (e.g., compound 14):

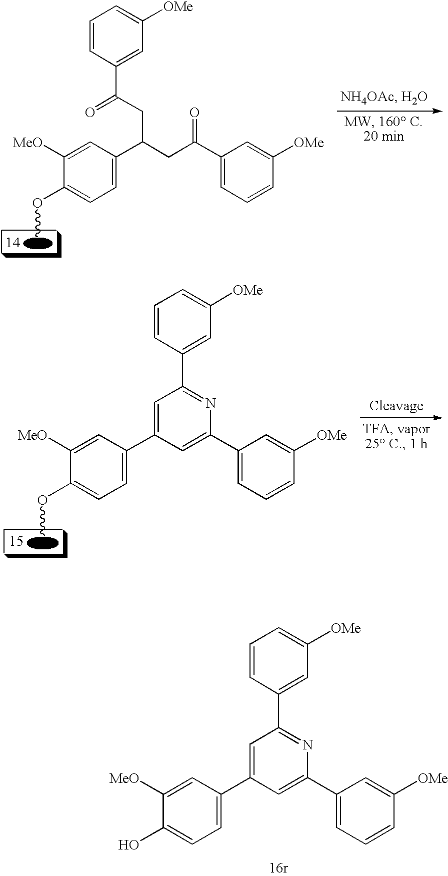

Ammonium acetate (NH₄OAc) has been used frequently in conjunction with acetic acid in this type of reaction; however, the acid-cleavable linker on the macroarray could be unstable under these conditions, especially at elevated temperatures. Instead, neutral, concentrated solution (3 M) of NH₄OAc in water was used for this condensation reaction.

The initial screen of MW-assisted reaction conditions to generate 2,4,6triarylpyridine 15 from support-bound diketone 14 revealed that the reaction proceeded best when the entire membrane was submerged in the aq. NH₄OAc solution, that is, under blanket-type reaction conditions. To examine reactions on small sections of support 14 (e.g., several punched-out spots), the reaction was performed in sealed 10 mL glass reaction vessels in a CEM Explorer monomodal MW system was the most convenient. These sealed-tube MW reaction conditions had two positive attributes: they permitted (1) automated temperature control during the MW reaction, and (2) heating over the boiling point of the solvent (i.e., 100° C. for water). The latter feature was important, as high temperatures were required for this reaction to proceed. Different temperatures were evaluated (120-180° C.) over 20 min reaction times. Reaction at 160° C. afforded the highest purity of the triarylpyridine product 16r product (82%), as compared to the 70% purity achieved at either 120° C. or 180° C. (as determined post-cleavage). The cellulose support was physically stable under these more forcing reaction conditions.

For the synthesis of full triarylpyridine macroarrays (40 spots), the standard, 10 mL glass MW reaction tubes were not large enough to accommodate the planar support (6 cm×15 cm, rolled into a tube). Instead, a 70 mL Teflon/polyetheretherketone (PEEK) reaction vessel proved more suitable for full macroarray synthesis in the Milestone MW multimodal reactor. In this vessel, reactions were performed in reusable Teflon inserts that fit inside the PEEK outer casing.

The temperature of the MW-assisted reaction could be controlled in this vessel using a fiber-optic probe threaded into the vessel. A ceramic sheath in the center of the vessel houses a fiber-optic probe for direct temperature measurement during the reaction.

In general, MW-assisted conditions directly translated from the monomodal to multimodal MW systems, if the reactions were performed under temperature control (Alcazar, J. J. Comb. Chem. 2005, 7, 353-355.) A minor change using a slower 10 min ramp time to 160° C. in the Milestone MW reactor relative to the CEM MW reactor was made in order to protect the integrity of the reusable Teflon inserts. The total reaction time was 1 h, consisting of a 10 min ramp, a 20 min hold time, and a 30 min cool down period (no MW) to 70° C., after which the vessel could be safely opened to retrieve the membrane.

This reaction was also examined under conventional heating conditions. Heating the sealed polymeric vessel to 160° C., however, was problematic for two reasons. First, the outer PEEK vessel acts as a highly efficient insulator; for example, in the MW-assisted triarylpyridine condensation reaction, the temperature of this outer vessel was measured to be only 120° C. (using a non-contact IR thermometer) even after the contents had been heated at 160° C. for 20 min (as determined using a fiber-optic probe). This insulation made it extremely difficult to mimic the MW temperature gradient inside the vessel using an external, conventional heating source (e.g., an oil bath). Matching heating gradients is a frequent challenge when performing comparisons of conventionally heated processes with MW-assisted variants (Kappe, C. O. Angew. Chem. Int. Ed. 2004, 43, 6250-6284.) Second, there are safety concerns about the prolonged conventional heating of closed vessels for macroarray synthesis. When membrane 14 was heated in aq. NH₄OAc in the oven at 80° C. for 1 h under atmospheric conditions, a lower conversion to triarylpyridine 16r (55% conversion) was observed. Complete conversion (>99%) and high purity product (89%) was obtained only after 12 h of heating at 80° C. in the oven. Therefore, application of MW heating to this condensation reaction significantly shortens reaction time compared to conventional heating. This benefit results, at least in part, because the MW reactor and specialized MW vessels allow for the safe and reproducible heating of solvents over their boiling points.

The MW-assisted method was used to construct a 40-member macroarray of symmetrical 2,4,6-triarylpyridines (16) using four hydroxybenzaldehyde and 10 acetophenone building blocks: 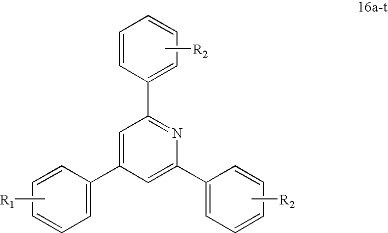
where R₁ and R₂ are listed in Table 4 below.

Starting from linker-derived support 104, the macroarray was synthesized and the array compounds were cleaved in only 12 h. Analysis of 20 randomly selected macroarray members (50% of the library) by LC-MS indicated good to excellent product purities (63-91%). Purities were largely substrate dependent, with the lowest purities occurring when a fluoro- or methoxy-substituent was in the para position of the incoming acetophenone. This reduction in purity may be due to positive mesomeric effects deactivating the chalcone for subsequent Michael addition. Two triarylpyridines 16l and 16r) were selected for synthesis in solution as controls in order to determine representative overall reaction yields on the triarylpyridine macroarray. Examination of calibration curves generated for 16l and 16r showed product yields of 78% and 88%, respectively, after cleavage of these compounds from the macroarray. These results demonstrate that macroarray synthesis represents a rapid and high yielding approach for the generation of symmetrical 2,4,6-triarylpyridines (16).

TABLE 4
Purity data for selected members of symmetrical 2,4,6-triarylpyridine
macroarray 16.

| entry | compound | R₁ | R₂ | purity (%)^{a} |
|---|---|---|---|---|
| 1 | 16a | 3-OH | 3-F | 85 |
| 2 | 16b | 3-OH | 4-F | 75 |
| 3 | 16c | 3-OH | 3-Br | 84 |
| 4 | 16d | 3-OH | 3-CF₃ | 85 |
| 5 | 16e | 3-OH | 4-CF₃ | 83 |
| 6 | 16f | 4-OH | H | 91 |
| 7 | 16g | 4-OH | 4-Br | 83 |
| 8 | 16h | 4-OH | 3-MeO | 86 |
| 9 | 16i | 4-OH | 3,4-MeO | 63 |
| 10 | 16j | 4-OH | 3-CF₃ | 85 |
| 11 | 16k | 4-OH, 3-OMe | 3-F | 76 |
| 12 | 16l | 4-OH, 3-OMe | 3-Br | 78 |
| 13 | 16m | 4-OH, 3-OMe | 4-Br | 84 |
| 14 | 16n | 4-OH, 3-OMe | 3-OMe | 90 |
| 15 | 16o | 4-OH, 3-OMe | 4-OMe | 63 |
| 16 | 16p | 3-OH, 4-OMe | 3-F | 75 |
| 17 | 16q | 3-OH, 4-OMe | 4-F | 67 |
| 18 | 16r | 3-OH, 4-OMe | 3-OMe | 88 |
| 19 | 16s | 3-OH, 4-OMe | 3-CF₃ | 73 |
| 20 | 16t | 3-OH, 4-OMe | 4-CF₃ | 74 |

^{a}Crude purity determined by integration of the HPLC trace with UV detection at 254 nm. Error ±3%.

Synthesis of Unsymmetrical Triarylpyridines.

Starting with support-bound acetophenones, rather than benzaldehydes, a far greater diversity of triarylpyridine products (23) could be achieved, as the products could be constructed in a step-wise manner. In an initial step, bound acetophenone building blocks (17) could be reacted with a series of benzaldehydes (18) to form chalcone macroarrays (19), using reaction conditions described above. In a subsequent step, the chalcones were treated with a second set of acetophenones to generate diketones (21). Conventional heating conditions were used in this step to achieve full conversion to the diketones (see above). Finally, diketones (21) could be condensed with NH₄OAc using our optimized MW-assisted conditions in the Milestone MW reactor to give triarylpyridine arrays (22). This step-wise synthesis is noteworthy, as it allows for control of the substituents on each phenyl ring of the triarylpyridine (23), and thus is amenable to the construction of triarylpyridine macroarrays (23) of high structural complexity.

Scheme 12. Synthesis of unsymmetrical 2,4,6-triarylpyridine macroarray 23:

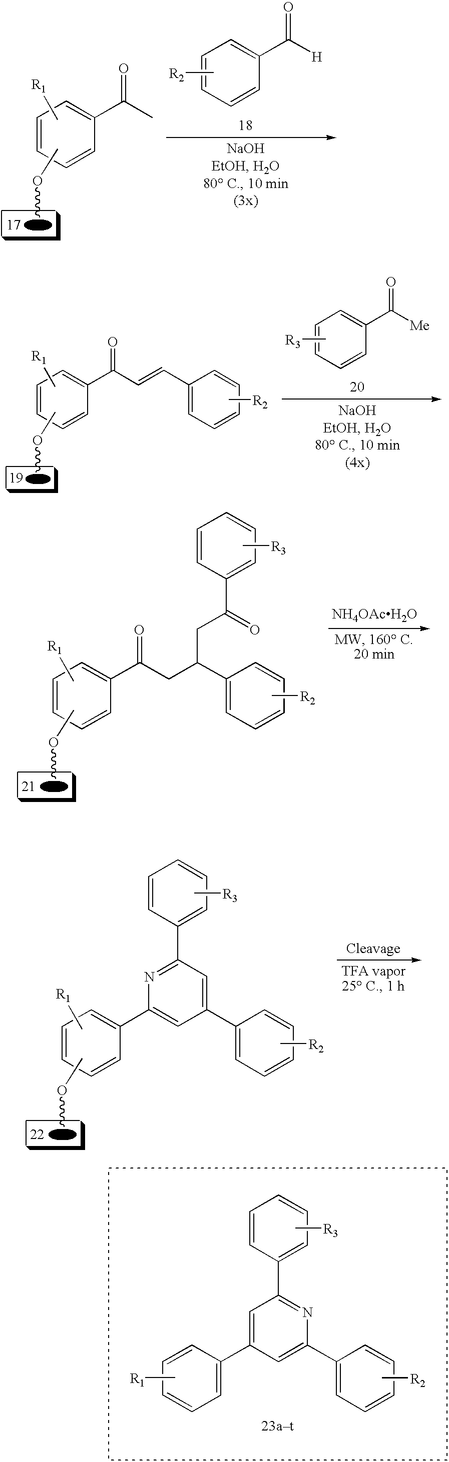

A 60-member macroarray of unsymmetrical 2,4,6-triarylpyridines (23):

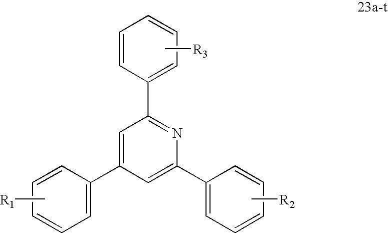

where representative R₁, R₂, and R₃ are listed in Table 5 below was synthesized using three hydroxyacetophenone (R₁=4-OH, 3-OH and 3-OMe with 4-OH), four benzaldehyde (R₂=H, 3-Br, 4-OMe, and 4-Cl), and five acetophenone (R₃=H, 4-Br, 4-CF₃, 3-CF₃, and 3-OMe) building blocks. Macroarrays of this size were readily constructed and cleaved in less than one day. Analysis of a random sampling of a third of the library members by LC-MS showed moderate to good purities (67-84%)

TABLE 5
Purity data for selected members of unsymmetrical 2,4,6-
triarylpyridine macroarray 23.

| | | | | | purity |
|---|---|---|---|---|---|
| Entry | compound | R₁ | R₂ | R₃ | (%)^{a} |
| 1 | 23a | 4-OH | H | 4-Br | 81 |
| 2 | 23b | 4-OH | H | 4-CF₃ | 79 |
| 3 | 23c | 4-OH | 3-Br | 3-CF₃ | 80 |
| 4 | 23d | 4-OH | 3-Br | 3-OMe | 75 |
| 5 | 23e | 4-OH | 4-OMe | 4-Br | 70 |
| 6 | 23f | 4-OH | 4-Cl | 3-OMe | 67 |
| 7 | 23g | 3-OH | H | 3-OMe | 72 |
| 8 | 23h | 3-OH | H | 3-CF₃ | 81 |
| 9 | 23i | 3-OH | 3-Br | 4-Br | 78 |
| 10 | 23j | 3-OH | 3-Br | 4-CF₃ | 76 |
| 11 | 23k | 3-OH | 4-OMe | H | 76 |
| 12 | 23l | 3-OH | 4-Cl | 4-Br | 76 |
| 13 | 23m | 3-OH | 4-Cl | 3-CF₃ | 80 |
| 14 | 23n | 3-OH | 4-OMe | 3-OMe | 74 |
| 15 | 23o | 4-OH, 3-OMe | H | 3-OMe | 70 |
| 16 | 23p | 4-OH, 3-OMe | 3-Br | 4-Br | 79 |
| 17 | 23q | 4-OH, 3-OMe | 3-Br | 4-CF₃ | 82 |
| 18 | 23r | 4-OH, 3-OMe | 4-OMe | H | 70 |
| 19 | 23s | 4-OH, 3-OMe | 4-OMe | 3-CF₃ | 77 |
| 20 | 23t | 4-OH, 3-OMe | 4-Cl | 3-CF₃ | 84 |

^{a}Crude purity determined by integration of the HPLC trace with UV detection at 254 nm. Error ±3%.

In specific embodiments, the methods herein can be employed to synthesis triaryl pyridines of formula 16, where R₁=R₂=R₃, R₁≠R₂≠R₃, R₁=R₂≠R₃, R₁≠R₂=R₃ and R₁=R₃≠R₂.

Overall, the ease of macroarray synthesis, along with the wide availability of numerous acetophenones and benzaldehydes, renders this method a powerful new technique for the rapid synthesis of 2,4,6-triarylpyridines.

Various alkylpyridine compounds are described in literature: Shibahashi, Yutaka; Sugai, Jun. “Reversible thermochromic composition for paints and inks,” Eur. Pat. Appl. (1995), EP 659582 A1; Finney, Nathaniel S.; Fang, Albert G.; Mello, Jesse V, “A preparation of arylpyridine derivatives, useful as fluorophores,” PCT Int. Appl. 2004) WO 2004046103 A2; Yarmoluk, Sergey M.; Kostenko, Olexandr M.; Tolmachev, Olexandr Iwanowitsch; Wolfbeis, Otto S, “Synthesis of fluorophore pyrylium derivatives and use as label for amino-group containing biomolecules and polymer particles,” Eur. Pat. Appl. (2003) EP 1308728 A2; Shibahashi, Yutaka; Sugai, Jun, “Reversible thermochromic composition for paints and inks,” Eur. Pat. Appl. (1995 EP 659582 A1; Geisler, Thomas C., “Stabilizer for electron donor-acceptor carbonless copying systems,” Eur. Pat. Appl. (1982) EP 62467 A2; Cationic dyes. Jpn. Kokai Tokkyo Koho (1981 JP 56004781 19810119 Showa; Siegrist, Adolf E.; Liechti, Peter; Maeder, Erwin; Guglielmetti, Leonardo; Meyer, Hans Rudolf; Weber, Kurt, “Ethylenically unsaturated heterocyclics,” Patentschrift (Switz.) (1973), CH 542212; Siegrist, Adolf E.; Liechti, Peter; Maeder, Erwin; Meyer, Hans Rudolf, “Fluorescent whiteners,” Patentschrift (Switz.) (1973)CH; Siegrist, Adolf E. “Distyrylbiphenyl fluorescent whitening agents,” Ger. Offen. (1972) DE 2148015; “Alkenic unsaturated heterocyclic compounds.” Neth. Appl. (1967), NL 6615211; Fang, A. G.; Mello, J. V.; Finney, N. S, “Structural studies of biarylpyridines fluorophores lead to the identification of promising long wavelength emitters for use in fluorescent chemosensors,” Tetrahedron (2004), 60(49), 11075-11087; Del Carmen, Maria; Barrio, G.; Barrio, Jorge R.; Walker, Graham; Novelli, Armando; Leonard, Nelson J., “2,4,6-Trisubstituted pyridines. Synthesis, fluorescence, and scintillator properties,” Journal of the American Chemical Society (1973), 95(15), 4891-5.

As noted above the method for synthesis of trialkylpyridines arrays of this invention is useful for creation of arrays of increased chemical complexity. In particularly, the triarylpyridines arrays and methods herein are useful for preparation of terpyridines and bipyridines, which have found widespread application in chemical sensors (Padilla-Tosta, M. E.; Lloris, J. M.; Martinez-Màñez, R.; Marcos, M. D.; Miranda, M. A.; Pardo, T.; Sancenón, F.; Soto, J. Eur. J. Inorg. Chem. 2001, 1475-1482) and organic light emitting diodes (Gao, F. G.; Bard, A. J. J. Am. Chem. Soc. 2000, 122, 7426-7427). These types of compounds can be synthesized readily using the methods herein through the incorporation of acetyl pyridine building blocks. The planar array format would facilitate a rapid screening of their sensing or photophysical properties while bound to the support.

The invention also provides kits detecting or labeling a biomolecule, for detection or measurement of pH, or for detection of a metal ion. The kits comprise a fluorescent compound of the invention suitable for the kit application. The kit can contain a planar support with one or more fluorescent compounds of this invention suitable for the application of the kit attached to the planar surface. Kits typically comprise components in suitable packaging and may contain a plurality of components suitable each suitable for a single use. Kit may further include instructions for carrying out one or more assays using the kit components. Kits may further comprise solvents, buffers, reagents, positive or negative controls for carrying out a given assay. The kit may be useful for labeling or detection of a nucleic acid, peptide, protein, or an antibody. The kit may be useful for labeling cellular compartments and organelles.

Unless defined otherwise, all technical and scientific terms used herein have the broadest meanings as commonly understood by one of ordinary skill in the art to which this invention pertains. The following definitions are provided.

As used herein, the term “array” refers to an ordered arrangement of structural elements, such as an ordered arrangement of individually addressed and spatially localized elements. Arrays useful in the present invention include arrays of containment structures and/or containment regions, such as fluid containment structures or regions, provided in a preselected, spatially organized manner. In some embodiments, for example, different containment structures and/or regions in an array are physically separated from each other and hold preselected materials, such as the reactants and/or products of chemical reactions.

Arrays of the present invention include “microarrays” and “macroarrays” which comprise an ordered arrangement of containment structures and/or containment regions capable of providing, confining and/or holding reactants, products, solvent and/or catalysts corresponding to one or more chemical reactions and reaction conditions. In some embodiments, a portion of the reactants and/or products confined in a containment structure/region of a microarray or macroarray are immobilized, for example by spatially localized conjugation to a selected region of containment structure or region. Microarrays and macroarrays of the present invention, for example, are capable of providing an organized arrangement of containment structures and/or regions, wherein different containment structures and/or regions are useful for providing, confining and/or holding preselected combinations of reactants having well defined and selected compositions, concentrations and phases. Containment structures and/or regions of microarrays and macroarrays are also useful for providing, confining and/or holding the products of chemical reactions. In some embodiments, for example, each containment structure and/or region of the microarrays and macroarrays is physically separated and contains the product of a different chemical reaction or a chemical reaction carried out under different reaction conditions.

The terms “microarray” and “macroarray” are used herein in a manner consist with the art. In some embodiments, a microarray comprises a plurality of containment structures or regions having at least one microsized (e.g., 1 to 1000s of microns) or sub-microsized (e.g., less than 1 micron) physical dimension. In some contexts, containment structures/regions of a microarray are smaller than containment structures/regions of a macroarray. In some contexts, containment structures/regions of a microarray are provided in a higher density than containment structures/regions of a macroarray. In some contexts, the number of containment structures/regions of a microarray is larger than the number of containment structures/regions of a macroarray. In specific embodiments, the invention provide macroarrays produced by SPOT synthesis are described herein and as known in the art. Macroarrays in the context of the present invention which are arrays of candidate compound for screening are prepared such that each compound member of the array (each spatially-localized compound) is present in an amount sufficient to allow its removal form the array for further analysis, for example, to measure spectral properties or to obtain confirmatory structural analysis (e.g., by mass spectroscopic analysis or NMR analysis).

The term “excitation wavelength” is a characteristic of a fluorophore, and refers to the wavelength of electromagnetic radiation capable of exciting fluorescence in a fluorophore or collection of fluorophores. Excitation wavelengths can be further characterized by a distribution of wavelengths of electromagnetic radiation which is capable of exciting fluorescence, commonly referred to as the fluorescence excitation spectrum. A distribution of excitation wavelengths may be characterized by one or more peak wavelengths corresponding to maxima in the distribution, and in some case by the full width at half maximum of the distribution. The distribution of excitation wavelengths of a fluorophore can vary with the chemical and physical environment of the fluorophore (e.g., solvent, pH, linking chemistry etc.).

The term “emission wavelength” is a characteristic of a fluorophore, and refers to the wavelength of fluorescence provided by a fluorophore or collection of fluorophores upon excitation. Emission wavelengths can be further characterized by a distribution of wavelengths of electromagnetic radiation corresponding to fluorescence from a fluorophore, commonly referred to as the fluorescence emission spectrum. A distribution of emission wavelengths may be characterized by one or more peak wavelengths corresponding to maxima in the distribution, and in some case by the full width at half maximum of the distribution. The distribution of emission wavelengths of a fluorophore can vary with the chemical and physical environment of the fluorophore (e.g., solvent, pH, linking chemistry etc.)

The term “Stokes shift” is a characteristic of a fluorophore which quantifies differences in the energies of electromagnetic radiation capable of exciting fluorescence and the energies of fluorescence from the fluorophore observed upon excitation. In some contexts, Stokes shift refers to the separation (or alternatively overlap) of the distribution of excitation wavelengths (e.g., excitation spectrum) and the distribution of emission wavelengths (e.g., the emission spectrum) for a given fluorophore. Stokes shift may be quantitatively characterized, for example, as the difference between the peak wavelength of the distribution of excitation wavelengths and the peak wavelength in the distribution of emission wavelengths. Fluorophores having a large Stokes shift are useful as probes in fluorescent labeling applications because the emission from these fluorophores can be more easily separated (e.g., optically filtered), detected and quantified than fluorophores having a small Stokes shift. The Stokes shift of a fluorophore can vary with the chemical and physical environment of the fluorophore (e.g., solvent, pH, linking chemistry etc.).

The term “quantum yield” is a characteristic of a fluorophore which quantifies the extent to which a fluorophore generates fluorescence upon excitation at a particularly wavelength. Quantum yield is quantitatively defined as:
(Quantum Yield)_{λ}=(No. of Emitted Photons)_{λ}/(No. of Absorbed Photons)_{λ};
Upon exposure of a fluorophore to electromagnetic radiation having a selected wavelength (λ). Quantum yields of a fluorophore have values equal to or less than 1, and vary as a function of wavelength. Fluorophores having larger quantum yields are useful as probes in fluorescent labeling applications because the emission from these fluorophores can be more easily detected and quantified than fluorophores having a smaller quantum yield. The quantum yield of a fluorophore can vary with the chemical and physical environment of the fluorophore (e.g., solvent, pH, linking chemistry etc.)
Chemical terms:

The term “alkyl” refers to a monoradical of a branched or unbranched (straight-chain or linear) saturated hydrocarbon and to cycloalkyl groups having one or more rings. Unless otherwise indicated preferred alkyl groups have 1 to 20 carbon atoms and more preferred are those that contain 1-10 carbon atoms. Short alkyl groups are those having 1 to 6 carbon atoms including methyl, ethyl, propyl, butyl, pentyl and hexyl groups, including all isomers thereof. Long alkyl groups are those having 8-20 carbon atoms and preferably those having 12-20 carbon atoms, as well as those having 12-20 and those having 16-18 carbon atoms. The term “cycloalkyl” refers to cyclic alkyl groups having preferably 3 to 20 carbon atoms having a single cyclic ring or multiple condensed rings. Cycloalkyl groups include, by way of example, single ring structures such as cyclopropyl, cyclobutyl, cyclopentyl, cyclohexyl, cyclooctyl, and the like, or multiple ring structures such as adamantanyl, and the like. Unless otherwise indicated alkyl groups including cycloalkyl groups are optionally substituted as defined below.

The term “alkenyl” refers to a monoradical of a branched or unbranched unsaturated hydrocarbon group having one or more double bonds and to cycloalkenyl group having one or more rings wherein at least one ring contains a double bond. Unless otherwise indicated preferred alkyl groups have 2 to 20 carbon atoms and more preferred are those that contain 2-10 carbon atoms, those having 2-6. Alkenyl groups may contain one or more double bonds (C═C) which may be conjugated or unconjugated. Preferred alkenyl groups are those having 1 or 2 double bonds and include omega-alkenyl groups. Short alkenyl groups are those having 2 to 6 carbon atoms including ethylene (vinyl), propylene, butylene, pentylene and hexylene groups including all isomers thereof. Long alkenyl groups are those having 8-20 carbon atoms and preferably those having 12-22 carbon atoms as well as those having 12-20 carbon atoms and those having 16-18 carbon atoms. The term “cycloalkenyl” refers to cyclic alkenyl groups of from 3 to 30 carbon atoms having a single cyclic ring or multiple condensed rings in which at least one ring contains a double bond (C═C). Cycloalkenyl groups include, by way of example, single ring structures (monocyclic) such as cyclopropenyl, cyclobutenyl, cyclopentenyl, cyclohexenyl, cyclooctenyl, cylcooctadienyl and cyclooctatrienyl as well as multiple ring structures. Unless otherwise indicated alkyl groups including cycloalkyl groups are optionally substituted as defined below.

The term “alkynyl” refers to a monoradical of an unsaturated hydrocarbon having one or more triple bonds (C≡C). Unless otherwise indicated preferred alkyl groups have 1 to 30 carbon atoms and more preferred are those that contain 1-22 carbon atoms. Alkynyl groups include ethynyl, propargyl, and the like. Short alkynyl groups are those having 2 to 6 carbon atoms, including all isomers thereof. Long alkynyl groups are those having 8-22 carbon atoms and preferably those having 12-22 carbon atoms as well as those having 12-20 carbon atoms and those having 16-18 carbon atoms. The term “cycloalkynyl” refers to cyclic alkynyl groups of from 3 to 30 carbon atoms having a single cyclic ring or multiple condensed rings in which at least one ring contains a triple bond (C≡C). Unless otherwise indicated alkynyl groups including cycloalkynyl groups are optionally substituted as defined below.

As used herein, the term “aryl” includes both carbocyclic and heterocyclic aromatic rings, both monocyclic and fused polycyclic, where the aromatic rings can be 5- or 6-membered rings. Representative monocyclic aryl groups include, but are not limited to, phenyl, furanyl, pyrrolyl, thienyl, pyridinyl, pyrimidinyl, oxazolyl, isoxazolyl, pyrazolyl, imidazolyl, thiazolyl, isothiazolyl and the like. Fused polycyclic aryl groups are those aromatic groups that include a 5- or 6-membered aromatic or heteroaromatic ring as one or more rings in a fused ring system. Representative fused polycyclic aryl groups include naphthalene, anthracene, indolizine, indole, isoindole, benzofuran, benzothiophene, indazole, benzimidazole, benzthiazole, purine, quinoline, isoquinoline, cinnoline, phthalazine, quinazoline, quinoxaline, 1,8-naphthyridine, pteridine, carbazole, acridine, phenazine, phenothiazine, phenoxazine, and azulene.

As used herein, aryl group also includes an arylalkyl group. Further, as used herein “arylalkyl” refers to moieties, such as benzyl, wherein an aromatic is linked to an alkyl group which is linked to the indicated position in the compound.

Alkyl, alkenyl, alkynyl and aryl groups may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl. A “hydroxy” group refers to an OH group. An “alkoxy” group refers to an —O-alkyl group wherein alkyl is as defined above. A “thio” group refers to an —SH group. A “thioalkyl” group refers to an —SR group wherein R is alkyl as defined above. An “amino” group refers to an —NH₂ group. An “alkylamino” group refers to an —NHR group wherein R is alkyl is as defined above. A “dialkylamino” group refers to an —NRR′ group wherein R and R′ are all as defined above. An “amido” group refers to an —CONH₂. An “alkylamido” group refers to an —CONHR group wherein R is alkyl is as defined above. A “dialkylamido” group refers to an —CONRR′ group wherein R and R′ are alkyl as defined above. A “nitro” group refers to an NO₂ group. A “carboxylic acid group refers to a COOH group.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure.

A number of specific groups of variable definitions have been described herein. It is intended that all combinations and subcombinations of the specific groups of variable definitions are individually included in this disclosure.

When a compound is described herein such that a particular isomer, enantiomer or diastereomer of the compound is not specified, for example, in a formula or in a chemical name, that description is intended to include each isomers, enantiomer or diastereomer of the compound described individually or in any combination.

Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Isotopic variants, including those carrying radioisotopes, may also be useful in diagnostic assays and in therapeutics. Methods for making such isotopic variants are known in the art. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein. Nothing herein is to be construed as an admission, however, that the invention is not entitled to antedate any disclosure by virtue of prior invention.

As used herein and in the appended claims, the singular forms “a”, “an”, and “the” include plural reference unless the context clearly dictates otherwise. Thus, for example, reference to “a compound” includes a plurality of such compounds. Additionally, the terms “a” (or “an”), “one or more” and “at least one” can be used interchangeably herein. As used herein and in the appended claims, “comprising” is synonymous with “including,” “having,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, reactants, reagents, reagents, synthetic methods, purification methods, analytical methods, assay methods, substrates, spectroscopic methods, biological materials and biological methods other than those specifically exemplified can be employed in the practice of the invention without resort to undue experimentation. All art-known functional equivalents, of any such materials and methods are intended to be included in this invention.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All references cited herein are hereby incorporated by reference herein in their entirety for any purpose. In general, publications mentioned herein are incorporated herein by reference for the purpose of describing and disclosing the chemicals, materials, instruments, methods of analysis including statistical analysis and methodologies which are reported in the publications which might be used in connection with the invention. Some references provided herein are incorporated by reference to provide details concerning sources of starting materials, additional starting materials, additional reactants, additional reagents, additional methods of synthesis, additional methods of analysis, additional spectroscopic methods, additional biological materials and additional uses of the methods and compounds of the invention.

### THE EXAMPLES

### Example 1

### Preparation of Macroarrays

General Methods. ¹H NMR and ¹³C NMR spectra were recorded on a Bruker AC-300 spectrometer in deuterated solvents at 300 MHz and 75 Hz, respectively. Chemical shifts are reported in parts per million (ppm, δ) using tetramethyl silane (TMS) as a reference (0.0 ppm). Couplings are reported in hertz. LC-MS (ESI) were obtained using a Shimadzu LCMS-2010 (Columbia, Md.) equipped with two pumps (LC-10ADvp), controller (SCL-10Avp), autoinjector (SIL-10ADvp), UV diode array detector (SPD-M10Avp), and single quadrupole analyzer (by electrospray ionization, ESI). The LC-MS is interfaced with a PC running the Shimadzu LCMS solution software package (Version 2.04 Su2-H2). A Supelco (Bellefonte, Pa.) 15 cm×2.1 mm C-18 wide-pore reverse phase column was used for all LC-MS work. Standard reverse phase HPLC conditions were as follows: flow rate=200 μL/min; mobile phase A=0.4% formic acid; mobile phase B=0.2% formic acid in acetonitrile. Attenuated total reflectance (ATR)-IR spectra were recorded with a Bruker Tensor 27 spectrometer, outfitted with a single reflection MIRacle Horizontal ATR by Pike Technologies. A ZnSe crystal with spectral range 20,000 to 650 cm⁻¹ was used. UV spectra were recorded using a Cary 50 Scan UV-Vis spectrometer running Cary WinUV 3.00 software. Fluorescence spectra were recorded on a Hitachi F-4500 Fluorescence spectrophotometer using the following parameters: 5.0 nm excitation slit width, 5.0 nm emission slit width, 1200 nm/min scan speed, 700 V PMT voltage, and 2.0 sec response time. Quantum yields were calculated using the method found in Lakowicz. Lakowicz, J. R. Principles of Fluorescence Spectroscopy. 2nd ed., New York: Kluwer Academic/Plenum Publishers, 1999. Thin layer chromatography (TLC) was performed on silica gel 60 F₂₅₄ plates (E-5715-7, Merck). Silica gel 60 (230-400 mesh, EM Science) was used for flash column chromatography. Still, W. C.; Kahn, M.; Mitra, A. J. Org. Chem. 1978, 43, 2923-2925. All reported melting points are uncorrected.

All reagents were purchased from commercial sources (Alfa-Aesar, Aldrich, and Acros) and used without further purification. Solvents were purchased from commercial sources (Aldrich and J. T. Baker) and used as is, with the exception of dichloromethane (CH₂Cl₂), which was distilled over calcium hydride immediately prior to use. Planar cellulose membranes (Whatman 1Chr chromatography paper, 20×20 cm squares) were purchased from Fischer Scientific and stored in a dessicator at room temperature until ready for use.

Microwave instrumentation. All microwave reactions were carried out in a Milestone MicroSYNTH Labstation multimodal microwave synthesis reactor equipped with a continuous power source (1000 W max), as shown in FIG. 13. This instrument is interfaced with an Ethos MicroSYNTH Lab Terminal PC running EasyWave reaction monitoring software. Using this reactor system, microwave irradiation can be applied to reactions using either power (wattage) control or temperature control. The microwave reactor is equipped with a fiber-optic temperature sensor that allows direct monitoring of the internal temperature of reaction vessels, and an infrared sensor (installed in the side wall of the reactor cavity) that can monitor the surface temperature of reaction vessels inside the cavity. For further information about this system, see: www.milestonesci.com/synth-micro.php. Solvent depths of ca. 1 cm in the reaction vessel are required for accurate temperature monitoring using the submerged fiber-optic temperature probe. Solution-phase reactions were performed in the MicroSYNTH reactor using specialized 70 mL Teflon/polypropylene reaction vessels. These vessels have appropriate holes in their lids to accommodate the fiber-optic temperature sensor in a protective ceramic sheath.

Cellulose supports were irradiated in a shallow Pyrex dish on a rotating turntable inside the MicroSYNTH microwave reactor. Planar supports were irradiated routinely using power control, as insufficient solvent volumes were used for accurate temperature monitoring using the fiber-optic probe.

Solid-phase synthesis techniques. Cellulose membranes were mixed with reagents at room temperature and washed with solvents in glass vessels on a Lab-Line orbital shaker housed in a fume hood. All spotting procedures used in SPOT-synthesis were performed manually using Brinkman Eppendorf pipettmen (calibrated for variable solvent delivery (1-10 μL)) and disposable polypropylene pipette tips. Frank, R. Tetrahedron 1992, 48, 9217-9232. Frank, R. J. Immunol. Methods 2002, 267, 13-26.

Representative planar cellulose membrane amination protocol. Dots were marked on a 14 cm×18 cm sheet of Whatman 1 Chr paper (1) at distances 1.4 cm apart using a #2 lead pencil. The sheet was immersed in 100 mL of 10% TFA in CH₂Cl₂ for 10 min in a covered 2.6 L Pyrex dish. This acid wash serves as a cellulose preactivation step and is believed to increase the surface area of the cellulose available for functionalization. Volkmer-Engert, R.; Hoffmann, B.; Schneider-Mergener, J. Tetrahedron Lett. 1997, 38, 1029-1032; Licha, K.; Bhargava, S.; Rheinlander, C.; Becker, A.; Schneider-Mergener, J.; Volkmer-Engert, R. Tetrahedron Lett. 2000, 41, 1711-1715. The sheets is washed by adding 60 mL of CH₂Cl₂, allowing it soak for 5 minutes, then decanting the CH₂Cl₂. This is repeated, then the membrane is dried under a stream of air. The sheet was then immersed in 50 mL of 2.0 M tosyl chloride in pyridine for 2 h. The paper was washed by immersion in three consecutive baths of ethanol (100 mL, 5 min each) and dried under a stream of nitrogen. The tosylated cellulose paper was immersed in 60 mL of neat 4,7,10-trioxa-1,13-tridecanediamine and placed on the rotating platform of the MicroSYNTH microwave reactor. Ast, T.; Heine, N.; Germeroth, L.; Schneider-Mergener, J.; Wenschuh, H. Tetrahedron Lett. 1999, 40, 4317-4318. The dish was covered with a second pyrex dish. The sheet was irradiated for 15 min at 400 W. The amine solution was carefully decanted from the paper. The paper was then washed by adding then decanting 70 mL portions of DMF, EtOH, 1.0N NaOH, H₂O, EtOH (2×), and CH₂Cl₂ (5 min in each bath). The amino paper was then dried under a stream of nitrogen.

Representative Fmoc quantitation protocol on cellulose supports. A SPOT (6 mm diameter) was punched from amino cellulose support using a desktop hole punch and immersed in 200 μL of 0.60 M Fmoc-OSu in DMF for 2 h. The SPOT was washed with 10 mL of ethanol, 10 mL of acetone, and 10 mL of CH₂Cl₂. After drying under a stream of nitrogen, 960 μL of DMF was added followed by 40 μL of 1,8-diazabicyclo[5.4.0]undec-7-ene (DBU). The mixture was swirled for 30 sec and then allowed to stand for 15 min. The mixture was swirled again for 30 sec, then 250 μL of this solution was removed and diluted with 1.0 mL of DMF. The solution was swirled again for 30 sec. The absorbance was read at 296 nm (ε₂₉₆=9500 M⁻¹ cm⁻¹) in a quartz cuvette. The value was multiplied by 5 to account for the dilution (Carpino, L. A.; Han, G. Y. J. Org. Chem. 1972, 37, 3404-3409). Loadings of 1.0-10 μmol/cm² were obtained using this method. Longer tosylation reaction times gave higher levels of functionalization (2 h=4.0 μmol /cm², 12 h=10 μmol /cm²).

Representative Wang-linker installation procedure on amino cellulose support. 4-formylphenoxyacetic acid (5.40 g, 30.0 mmol), diisopropylcarbodiimide (DIC, 4.7 mL, 30.0 mmol), N-hydroxysuccinimide (3.45 g, 30.0 mmoles), NEt₃ (4.2 mL, 30.0 mmol), and DMF (50 mL) were combined in a 2.6 L Pyrex dish. The dish was covered and swirled for 30 min at room temperature. A 14 cm×18 cm sheet of amino cellulose paper (3) was added. The dish was covered again and the mixture was swirled at room temperature for 2 h. The coupling solution was decanted. The decanted linker solution can be poured onto a second sheet of aminocellulose. Immersion overnight in the solution will give a sheet with similar loadings to the primary sheet. The decanted linker solution can be poured onto a second sheet of aminocellulose. Immersion overnight in the solution will give a sheet with similar loadings to the primary sheet. The paper was then washed by adding then decanting 70 mL portions of DMF (2×), EtOH (2×), and CH₂Cl₂ (5 min in each bath). The aldehyde-derived paper (5) was then dried under a stream of nitrogen.

100 mL of 1.0 M NaBH₄ in 1.0 M aq. NaOH was added to the aldehyde-derived support 5. The mixture was swirled for 20 min. The NaBH₄ solution was decanted. The paper was then washed by adding then decanting 70 mL portions of H₂O (2×), EtOH (2×), and CH₂Cl₂ (5 min in each bath). The benzyl alcohol paper (6) was dried under a stream of nitrogen. To approximate the linker loading, the amount of residual amine was measured by Fmoc quantitation as described above, except a 2-3-fold dilution was used instead. Residual amine loadings are ca. 600-800 nmol/cm².

Representative Wang-linker activation protocol. Immediately prior to use, a 14 cm×18 cm sheet of benzyl alcohol paper (6) was activated by submerging it in 50 mL of a 4.0 M solution of tosyl chloride in dry DMF and swirled for 30 min on an orbital shaker. The paper was then washed by immersion and swirling in dry CH2Cl2 (70 mL, 3×2 min). The benzyl chloride-derived paper was then dried by a stream of nitrogen.

Cellulose membranes were mixed with reagents at room temperature and washed with solvents in glass vessels on a Lab-Line orbital shaker housed in a fume hood. All spotting procedures used in SPOT-synthesis were performed manually using Brinkman Eppendorf pipettmen (calibrated for variable solvent delivery (1-10 μL) and disposable polypropylene pipette tips.

Each spot on the macroarray is typically about 0.3 cm² in area and affords approximately 100 nmoles of compound. This scale has been found to facilitate routine array construction. The SPOT-Synthesis of a chalcone and fluorescent dye macroarrays as performed as follows.

### Example 2

### SPOT-Synthesis of Chalcone Macroarray (1)

Coupling of hydroxyacetophenones to activated planar support. A 2.0 M coupling solution of the various hydroxyacetophenones solutions were prepared by adding an equal volume of a 4.0 M solution of KOtBu in anhydrous DMF to a 4.0 M solution of substituted hydroxyacetophenone (7) in anhydrous DMF. Twelve 3.0 μL aliquots of each solution was applied to activated Wang-linker derived cellulose as twelve SPOTs at distances 1.4 cm apart along three rows (A-B):

| | |
|---|---|
| Row A: | 4′-hydroxyacetophenone |
| Row B: | 3′-hydroxyacetophenone |
| Row C: | acetovanillone |

The planar support was then subjected to microwave irradiation in a Pyrex dish at 500 W for 10 min in the MicroSYNTH microwave reactor. The support was next washed in a low Pyrex dish by adding then decanting 70 mL portions of 1N NaOH, H₂O (2×), EtOH (2×), and CH₂Cl₂ (5 min in each bath). The acetophenone-derived support (8) was dried under a stream of nitrogen.

Claisen-Schmidt condensation on planar support. The following benzaldehyde solutions were prepared and three aliquots were spotted onto acetophenones-derived support at distances 1.4 cm apart down the corresponding columns (1-12):

| | |
|---|---|
| Column 1: | 6.0 μL of 1.0 M benzaldehyde in 1.5 N NaOH in 50% aq. EtOH. |
| Column 2: | 6.0 μL of 0.5 M 2-fluorobenzaldehyde in 1.5 N NaOH in 50% aq. EtOH |
| Column 3: | 6.0 μL of 1.0 M 3-fluorobenzaldehyde in 1.5 N NaOH in 50% aq. EtOH |
| Column 4: | 6.0 μL of 1.0 M 4-fluorobenzaldehyde in 1.5 N NaOH in 50% aq. EtOH |
| Column 5: | 6.0 μL of 0.5 M 3-bromobenzaldehyde in 1.5 N NaOH in 50% aq. EtOH |
| Column 6: | 6.0 μL of 0.5 M 4-bromobenzaldehyde in 1.5 N NaOH in 50% aq. EtOH |
| Column 7: | 6.0 μL of 0.5 M 4-chlorobenzaldehyde in 1.5 N NaOH in 50% aq. EtOH |
| Column 8: | 6.0 μL of 1.0 M 3,4-difluorobenzaldehyde in 1.5 N NaOH in 50% aq. EtOH |
| Column 9: | 6.0 μL of 0.5 M m-anisaldehyde in 1.5 N NaOH in 50% aq. EtOH |
| Column 10: | 6.0 μL of 1.0 M p-anisaldehyde in 1.5 N NaOH in 50% aq. EtOH |
| Column 11: | 6.0 μL of 1.0 M 3-chlorobenzaldehyde in 1.5 N NaOH in 50% aq. EtOH |
| Column 12: | 6.0 μL of 1.0 M 4-dimethylaminobenzaldehyde in warm ethylene glycol |

To column 12, 8.0 uL of 5.0 M KOtBu in ethylene glycol was added. The support was placed in a Pyrex dish in the MicroSYNTH microwave reactor and irradiated at 400 W for 10 min. Columns 1-12 were then respotted with the benzaldehyde solutions and irradiated a second time in the MicroSYNTH microwave reactor at 400 W for 10 min. The support was next washed in a low Pyrex dish by adding then decanting 70 mL portions of 1% aq. AcOH, DMSO, EtOH (2×), and CH₂Cl₂ (5 min in each bath). The chalcone-derived support was dried under a stream of nitrogen.

### Example 3

### SPOT-Synthesis of Fluorescent Dye Macroarrays

Cyanopyridine Fluorescent Dye Macroarray (2): KOtBu (1.234 g, 11.0 mmol) was suspended in 5.0 mL of acetonitrile in a 20 mL vial. The mixture was capped with a teflon cap and sonicated in an ultrasound bath (Branson model # 151OR-MT) for 50 minutes. After sonication, 6.0 uL of the mixture was applied to each SPOT on a chalcone array 1. The membrane was allowed to stand and room temperature for ten minutes. The spotting and standing steps were repeated three more times. The membrane was next washed in a low Pyrex dish by adding then decanting 70 mL portions of DMSO, 1% aq. AcOH, EtOH (2×), and CH₂Cl₂ (5 min in each bath). The cyanopyridine array was then dried under a stream of nitrogen.

Deazalumazine Fluorescent Dye Macroarray (3): 6-amino-2,3-dimethyl uracil (77.6 mg, 0.5 mmol) was dissolved in 1.5 mL of DMSO and 0.5 mL of 1.0 N aq. NaOH. 6.0 uL of this solution was applied to each SPOT on a chalcone array 1. The support was placed in a Pyrex dish in the MicroSYNTH microwave reactor and irradiated at 400 W for 10 min. The spotting and microwaving steps were repeated three more times. The membrane was next washed in a low Pyrex dish by adding then decanting 70 mL portions of DMSO, 1% aq. AcOH, EtOH (2×), and CH₂Cl₂ (5 min in each bath). The deazalumazine array was then dried under a stream of nitrogen.

Vapor -phase cleavage of dye macroarrays. This cleavage method is a modified version of that reported recently by Scharn et al. (Scharn, D., et al., J. Comb. Chem. 2000, 2, 361-369). The SPOTs were punched out and placed in individual vials. 10 mL of TFA was added to the bottom of a vacuum dessicator. A Petri dish was placed above the TFA and the vials containing the library members were placed on that. The dessicator was evacuated to 60 mbar for 10 min and then sealed for 50 min. The seal was broken, and the SPOTs were then taken out of the dessicator. After standing in a fume hood for 30 minutes, 1.0 mL of ethanol was added to each vial. After swirling for 15 minutes, 0.5 mL was saved for LC-MS analyses in separate glass vials, and the remaining 0.5 mL were used for spectral characterization.

LC-MS analysis of dye macroarrays. The ethanol solutions were concentrated under reduced pressure using a SpeedVac centrifuge system. The resulting residues were dissolved in 150 μL of 50% aqueous acetonitrile, filtered through a cotton plug, and analyzed by LC-MS. HPLC: conditions as described above, solvent gradient 20-95% B in 8 min followed by 4 min at 95% B. MS: (ESI) positive ion mode was used for all samples. Relative conversions (in comparison to acetophenone and chalcone starting materials) and compound purities were determined by integration of peaks in the HPLC trace at 254 nm using the Shimadzu LCMSsolution software package (Version 2.04 Su2-H2).

### Example 4

### Improvements to SPOT-Synthesis Process

Linker Derivatization. An acid-cleavable (Wang-type) linker-derivatized support is used in macroarray synthesis (Wang, S. S. J. Am. Chem. Soc 1973, 95, 1328-1333). As originally reported, this reaction sequence took over 21 h and was a considerable bottleneck in macroarray construction (Bowman, M. D.; Jeske, R. C.; Blackwell, H. E. Org. Lett. 2004, 6, 2019-2022). The overall procedure for preparation of linker-derivatized support is outlined in Scheme 1. The first step was a pre-swelling of the cellulose support (20 cm×20 cm sheet) using trifluoroacetic acid (TFA) to increase the surface area and thus the number of reactive sites for derivatization. Doubling the concentration of TFA in the initial swelling step from 10% to 20% resulted in better reproducibility in linker loading levels. Removing the residual TFA from the support prior to subsequent steps, however, required lengthy drying times in a vacuum oven (over 16 h). As an alternative to vacuum drying, two 5-min washes of anhydrous CH₂Cl₂ and a 20 min drying time using a stream of N₂ gave a support of equal quality. Notably, this simple procedural adjustment significantly reduced the synthesis time (by 15 h).

The pre-swelled paper was originally reacted with tosyl chloride (TsCl) in pyridine (2.0 M) at room temperature to generate tosylated cellulose support. Treating the support with TsCl solution for variable amounts of time gave different and reproducible tosylation levels (1 h˜3.8 μmol/cm², 10 h˜10.0 μmol/cm², as determined via subsequent amination reactions). Amine functionalization levels were determined by derivatization of support 103 with Fmoc-OSu, cleavage of the N-Fmoc group with piperidine, and quantitation of released fulvene product using UV spectroscopy (at 296 nm) according to standard procedures. See: Carpino, L. A.; Han, G. Y. J. Org. Chem. 1972, 37, 3404-3409.

A flexible diamino “spacer” unit, 4,7,10-trioxatridecanediamine, was then introduced onto support the tosylated support through standard nucleophilic substitution chemistry (FIG. 1). This “spacer” unit has been shown to improve the accessibility of array-bound molecules for subsequent reactions and on-support assays. The spacer can be coupled to the tosylated support by either MW-assisted conditions (400 W, 15 min in a Milestone MicroSYNTH Labstation multimode MW reactor) or conventional heating in an oven (80° C., 30 min) to achieve equivalent amine loadings (ca. 4-10 μmol/cm²). Thus, use of the MW reactor in this displacement step provided only a minor reduction in synthesis time.

Wang-linker derivatized support can be prepared from amino support using two different, yet complimentary, methods. Both methods involve two stages: first, the “pro-linker”, 4-formylphenoxyacetic acid, was coupled to amino support 103 via a standard carbodiimide (DIC) coupling at room temperature. After which the resulting aldehyde support was reduced using sodium borohydride (NaBH₄) at room temperature to yield benzylalcohol-derived support. The two methods only differed in how the pro-linker was physically applied to the planar support in the first step. In the first method, the entire membrane was immersed in the pro-linker coupling solution for 2 h in a “blanket-type” functionalization. This method gave the highest linker loading overall (2.6 μmol/cm²). However, this bulk procedure required a large quantity of pro-linker reagent, and as such does not represent a general linker loading strategy if the linker reagent is either expensive or difficult to synthesize. In the second method, the pro-linker was applied in a spatially addressed, or “spotted”, manner, delivering 6.0 μL aliquots of the activated solution to individual spots on amino support 103 (applied twice over 2 h). Spatially addressed delivery only required 20% of the quantity of linker relative to the blanket-type method; however, linker loadings were diminished (1.8 μmol/cm²). Since 4-formylphenoxyacetic acid is readily available either synthetically or from commercial sources, the blanket-type functionalization technique was chosen to generate support 104 throughout the present study. Using this optimized method, four 20 cm×20 cm sheets of linker-derived support 104, each with 120 spot-capacity, were routinely prepared in under 8 h. This represents a significant improvement in synthesis efficiency, as the original method required over 21 h to generate a single sheet of support 104. The sheets prepared by these methods were found to be stable at room temperature for at least one month (stored in a dessicator) and could be used in an “off-the-shelf” manner for small molecule macroarray construction.

Benzylic alcohol-derived support 104 requires activation prior to the attachment of macroarray building blocks. Conversion of support 104 to the benzylic chloride 105 was found to be an effective activation approach (Scheme 1). In initial work, chlorination of support 104 was achieved with TsCl in anhydrous DMF. This method was extremely sensitive to the level of moisture in the DMF solvent. Chlorination with SOCl₂ in anhydrous CH₂Cl₂ gives comparable chlorination levels to the original method (based on the loading of 4′-hydroxyacetophenone (6), see below), yet is far more reproducible. This may be due to the relative ease of maintaining an anhydrous environment with CH₂Cl₂. After chlorination, activated membranes 105 were stored under N₂ and had to be submitted promptly to macroarray construction (within 15 min of preparation) in order to minimize hydrolysis.

Optimization of Initial Building Block Loading. The loading of hydroxyacetophenone building blocks onto activated support 105 was examined under a variety of conventional and MW heating conditions to determine if MW-assisted reactions provided an advantage for this step in macroarray construction. 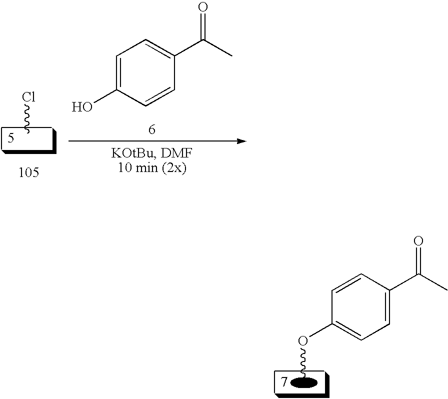
4′-hydroxyacetophenone (6) was used as a model substrate, and identical conditions were used for all reactions to facilitate direct comparison between the two heating methods (3.0 □l aliquot of 2.0 M 6 and 2.0 M KOtBu in anhydrous DMF, 10 min). Application of this volume of reagent to support 105 provided a spot with a 0.3 cm² area. Conventional heating conditions were examined in a standard laboratory drying oven (VWR model #13OOU, for ≧80° C.) or a laboratory incubator (Lab-Line Imperial II, for 40° C. reactions). MW heating conditions for this step were examined in a Milestone MicroSYNTH multimodal MW reactor. The temperature of the planar support surface could be measured easily during these reactions using a non-contact IR thermometer (Craftsman model # 82327) positioned in apertures at the top of either the oven or MW reactor.

Heating conditions tested included:

TABLE 6

| conditions (final | membrane substrate |
|---|---|
| temperature) | loading (nmol/cm²)^{a} |
| room temperature (22° C.) | 60 |
| oven (40° C.)^{b} | 80 |
| oven (80° C.) | 260 |
| MW 500 W (40° C.)^{c} | 120 |
| MW 500 W, on Pyrex dish (80° C.)^{d} | 120 |
| oven (80° C.)^{e} | 190 |

^{a}Determined by integration of the HPLC trace with UV detection at 254 nm. Integration values were compared to a UV calibration curve generated for 6. Error ±10 nmol/cm².
^{b}Performed in a laboratory incubator.
^{c}Support suspended in the middle of the MW reactor cavity using tape to minimize heating due to conduction from a surface.
^{d}Support placed in flat 2.6 L Pyrex dish and then on rotor in MW cavity. Pyrex dish thickness = 0.5 cm.
^{e}Prepared using support 4 on which the linker was applied in a spatially addressed manner.

The use of a drying oven could provide inconsistent heating of the planar supports. Depending on the position in the oven, temperature could vary easily by up to ±10° C. This made the uniform heating of 20 cm×20 cm planar support sections in the oven difficult, especially over short times (e.g., 5-10 min). This homogeneity problem was solved by placing a large sand bath in the oven that was allowed to equilibrate to the target temperature overnight prior to a conventional heating experiment. The sand bath was found to give stable and reproducible temperatures that varied only by ±1° C. The planar supports were heated by simply placing them on top of the sand. The sand bath provided an added benefit to conventional heating in the oven, since it dramatic minimized temperature changes that accompanied the opening and closing of the oven door.

The loading of acetophenone 6 increased steadily with increasing temperature using conventional heating methods, with the highest achievable loading obtained at 80° C. The original heating method using the MW oven,, however, resulted in an intermediate loading level relative to conventional heating, even when the final temperature of the membrane also reached 80° C. (entry 5). The rapid heat transfer observed from the pre-heated sand bath in the oven to the support could in part explain this difference. After 30 sec of heating on the sand bath, the temperature of the support was measured to be 80° C. In contrast, the temperature of the Pyrex dish used in the MW-assisted reaction only reached 80° C. (from room temperature) after 20 min of constant irradiation in the MW reactor (500 W).

Interestingly, the loading values achieved when the support was heated either in or outside of a Pyrex dish in the MW reactor were equivalent. This suggests that a different heating mechanism than simple conduction could be operative for these MW-assisted reactions. Quantifying such a heating mechanism is difficult using bulk temperature measurements such as IR, as often these measurements do not reflect microenvironments of higher temperatures in a material (or “hotspots”). The presence of such hotspots could explain the higher loading achieved using the MW reactor (without the Pyrex dish) versus heating in the oven at 40° C., even though the bulk temperatures of the membranes were measured to be identical. Finally, lower hydroxyacetophenone loadings are obtained when the pro-linker is applied in a spatially addressed manner compared to in a blanket-type functionalization (see above).

This study revealed that heating in an 80° C. oven was the highest yielding method for initial hydroxyacetophenone substrate attachment. It should be noted, however, that this procedure only gave hydroxyacetophenone loading values equal to 10% of the available reactive linker sites (260 nmol/cm² vs. 2.6 μmol/cm²). Repetitive applications of reagents and heating failed to show a significant improvement in loading for this reaction. This may be caused by reaction of the benzylic chlorides of activated support 5 with residual water in the membrane or with free hydroxyls on the cellulose surface as opposed to substrate 6. The use of non-anhydrous DMF or older bottles of KOtBu has been observed to reduce loading. Alternatively or additionally, native hydroxyl groups on the cellulose paper may be activated during the chlorination protocol and compete with the activated linker for substrate. Evidence for this latter theory was obtained when fluorescent hydroxyacetophenones were coupled to 5 and repeated exposure to cleavage and elution conditions failed to remove the substrates completely from the surface (i.e., fluorescence was observed when the membranes were irradiated with UV light.)

Optimization of the Claisen-Schmidt Condensation. Claisen-Schmidt condensation proceeds smoothly under MW-assisted conditions on planar support-bound acetophenones (400 W, 20 min). A systematic comparison of the MW-assisted condensation reaction was made to condensation performed under conventional heating, using the analogous drying oven conditions and non-contact temperature measurements described above for acetophenone loading. Support-bound acetophenone was selected as the substrate for optimization studies.

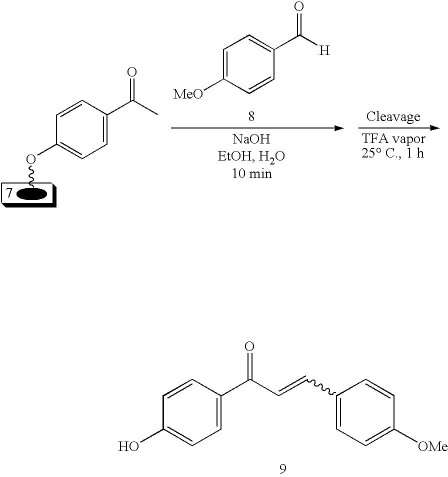

A wide variety of substituted benzaldehydes were reactive with support-bound acetophenone under the following spatially addressed, Claisen-Schmidt condensation conditions: 6.0 μL aliquot of 1.0 M benzaldehyde and 1.5 N NaOH in 50% aq. EtOH, 10 min. p-Anisaldehyde was chosen as a condensation partner, for further optimization studies, as this substrate was found to react to give chalcone product at a rate that was convenient for repeated analyses (e.g., entry 1, 22% conversion to chalcone after 10 min at room temperature). Again, macroarray spots of 0.3 cm² area were examined. Conversion and yield of chalcone were determined after TFA vapor compound cleavage from the support.

Results of varying conditions of Claisen-Schmidt condensation of support-bound acetophenone 7 are:

TABLE 7

| | | conversion | purity of | yield of |
|---|---|---|---|---|
| entry | conditions (final temperature) | (%)^{a} | 9 (%)^{b} | 9 (%)^{c} |
| 1 | room temperature (22° C.) | 22 | 13 | 16 |
| 2 | oven (40° C.)^{d} | 46 | 29 | 30 |
| 3 | oven (80° C.) | 81 | 62 | 48 |
| 4 | oven (120° C.) | 90 | 76 | 59 |
| 5^{e} | oven (80° C.) | 95 | 83 | 72 |
| 6^{e} | oven (120° C.) | 97 | 69 | 49 |
| 7 | MW 500 W (40° C.)^{f} | 45 | 26 | 26 |
| 8 | MW 500 W Pyrex dish (70° C.) | 47 | 24 | 23 |
| 9^{g} | MW 500 W Pyrex dish (80° C.) | 92 | 81 | 62 |
| 10^{g} | oven (80° C.) | >99 | 97 | 87 |

^{a}Based on residual 6 observed in HPLC spectra and quantified using a UV calibration curve at 254 nm. Error ±3%.
^{b}Determined by integration of HPLC spectra with UV detection at 254 nm. Error ±3%.
^{c}Quantified using a UV calibration curve at 254 nm. Error ±5%.
^{d}Performed in a laboratory incubator.
^{e}Anisaldehyde (8)/base solution was applied a second time and the support was heated for an additional 10 min.
^{f}Support suspended in the middle of the MW reactor cavity using tape to minimize heating due to conduction from a surface.
^{g}Anisaldehyde (8)/base solution was applied a third time and the support was heated for an additional 10 min.

Similar to observations for acetophenone 6 loading, conversion to chalcone product 9 increased steadily with increasing temperature using conventional heating and one application of anisaldehyde (8) (entries 1-4). Application of a second aliquot of anisaldehyde (8) and heating again at either 80° C. or 120° C. was observed to increase conversion (entries 5 and 6); however, substantial byproducts appeared and the yield of chalcone 9 was diminished at the higher temperature. MW-assisted conditions (500 W) with one application of anisaldehyde (8) (entry 7) gave similar conversions to chalcone product 9 as conventional heating in the oven at 40° C. (entry 2). Heating to 40° C. using either the MW reactor or oven gave similar results. Again, use of the Pyrex dish did not significantly impact conversion for the MW-assisted reaction (entry 8). Analogous to conventional heating, a double application of anisaldehyde (8) in the MW-assisted reaction gave a marked increase in the product conversion (entry 9) that was comparable to double coupling and conventional heating at 80° C. (entry 5). However, the yield of chalcone 9 was slightly reduced. Finally, careful optimization of both heating methods revealed that spotting the anisaldehyde (8) solution and heating at 80° C. in the oven three times gave the highest yield and purity of chalcone product 9 (entry 10). Conventional heating methods for the Claisen-Schmidt condensation therefore appear slightly superior for this step in macroarray synthesis.

The use of hydroxybenzaldehydes, in contrast to hydroxyacetophenones, as the planar support-bound substrate in the Claisen-Schmidt condensation was investigated. This approach would be attractive because a substantially larger number of substituted hydroxybenzaldehyde building blocks are commercially available relative to substituted hydroxyacetophenones, and could permit the construction of chalcone macroarrays with increased structural complexity. It was observed that the loading conditions for hydroxyacetophenones described above were directly translatable to hydroxybenzaldehydes, achieving analogous compound loadings (ca. 290 nmol/cm²). However, during the subsequent Claisen-Schmidt condensation step between support-bound hydroxybenzaldehydes and solution-phase acetophenones, two competing reaction pathways were observed.

### Example 5

### Synthesis of Triarylpyridines

Representative synthesis of chalcone macroarray (19). A ca. 300-500 μL solution of substituted benzaldehyde (18, 1.0 M) and NaOH (1.5 N) was prepared in 50% aq. EtOH in a 4 mL vial and sealed with a Teflon cap. (A 0.5 M solution of substituted benzaldehyde (18) in 1.5 N NaOH in 50% aq. EtOH can also be substituted in cases where preparing a 1.0 M solution of benzaldehyde substrates is impossible due to solubility reasons. For example, 4-chlorobenzaldehyde and m-anisaldehyde require these alternate conditions). A 6.0 μL aliquot of this solution was applied to the appropriate spots on a 15 cm×18 cm sheet of hydroxyacetophenone macroarray 17. The support then was placed on a bed of pre-heated sand in a drying oven set to 80° C. for 10 min. The spotting and heating steps were repeated (2×). The support was removed and washed by adding and decanting 100 mL portions of 1% aq. AcOH, DMSO, EtOH (2×), and CH₂Cl₂ (5 min in each wash). The resulting chalcone macroarray (19) was dried under a stream of air for 20 min.

Representative synthesis of diketone macroarrays (15 and 22). A ca. 300-500 μL solution of substituted acetophenone (1.0 M) and NaOH (1.5 N) was prepared in 66% aq. EtOH in a 4 mL vial and sealed with a Teflon cap. (A 0.5 M solution of acetophenone in 1.5 N NaOH in 66% aq. EtOH can be substituted in cases where preparing a 1.0 M solution of acetophenone substrate is impossible due to solubility reasons. For example, 4′-bromoacetophenone and 3′-bromoacetophenone require these alternate conditions). A 6.0 μL aliquot of this solution was applied to the appropriate spots on hydroxybenzaldehyde macroarray 10 to generate symmetrical diketones or chalcone macroarray 19 to generate unsymmetrical diketones. The support was placed on a bed of pre-heated sand in the oven set to 80° C. for 10 min. The spotting and heating steps were repeated (3×). The membrane was removed and washed by adding and decanting 100 mL portions of 1% aq. AcOH, DMSO, EtOH (2×), and CH₂Cl₂ (5 min in each wash). The resulting symmetrical diketone (15) and unsymmetrical diketone macroarrays (22) were dried under a stream of air for 20 min.

Representative synthesis of triarylpyridine macroarrays (16 and 23). A 3.1 M stock solution of aq. NH₄OAc was prepared by dissolving NH₄OAc (77.08 g, 1.0 mol) in 250 mL of water. A dried diketone macroarray (15 or 22) measuring 15 cm×6 cm (40 spots) was gently rolled into a tube and placed inside a 70 mL Teflon/PEEK MW reaction vessel. A 50 mL portion of the aq. NH₄OAc solution was added to the vessel, and the vessel was sealed. Using this method, multiple macroarrays could be prepared simultaneously using multiple Teflon/PEEK reaction vessels. The vessels were placed on a rotating base inside the Milestone MW reactor, and the fiber-optic probe was introduced into one of the vessels. Using a maximum wattage of 800 W, the reaction mixtures were heated from room temperature to 160° C. over 10 min, held at 160° C. for 20 min, and allowed to cool for 30 min. The supports were removed, unrolled, and washed by adding and decanting 100 mL portions of H₂O, EtOH (2×), and CH₂Cl₂ (5 min in each wash). The resulting triarylpyridine macroarrays (16 and 23) were dried under a stream of N₂ for 20 min.

### Example 6

### Solution-Phase Synthesis of Precursors

1-(4-(tetrahydro-2H-pyran-2-yloxy)phenyl)ethanone. 4-hydroxyacetophenone (8.5 g, 62.5 mmol), 3,4-dihydropyran (8.5 mL, 100 mmol), and p-toluenesulfonic acid (0.5 g 5.0 mmol) were dissolved in 125 mL of dichloromethane equipped with a stirbar and stirred for 17 hours at room temperature. The reaction mixture was washed with saturated aq. Na₂CO₃, the organic layer was dried over MgSO₄, and concentrated under reduced pressure to afford 12.86 g of pale brown solid. 93% yield. Characterization matched that reported by Ma and Venanzi. Ma, S.; Venanzi, L. M. Tetrahedron Lett. 1993, 34, 5269-5272.

Solution-Phase Synthesis of Chalcone Precursors

4-dimethylamino-4′-(tetrahydro-2H-pyran-2-yloxy)chalcone. 1-(4-(tetrahydro-2H-pyran-2-yloxy)phenyl)ethanone (2.64 g, 12.0 mmol), 4-dimethylaminobenzaldehyde (1.80 g, 12.0 mmol) were dissolved in ethanol (40 ml) in a 100 mL round bottom flask. 50% (w/v) aqueous sodium hydroxide (2 ml) was added, and the solution was stirred overnight (approximately 18 hours) at room temperature. The yellow precipitate was filtered and recrystallized from methanol to afford 3.3 g of yellow-orange solid. 78% yield. TLC R_{f}=0.31 (20% ethyl acetate in hexane); Melting point: 110-111° C.; ¹H-NMR (300 MHz, DMSO-d6)8.09, 7.13 (AA′XX′, J_{AA′}=J_{XX′}=2.4, J_{AX}=8.7, J_{AX′}=0.2 Hz, 4H), 7.63 (m, 2H), 7.61 (d, J=1.3 Hz), 6.72 (m, 2H), 5.6 (m, 1H), 3.73 (m, 1H), 3.59 (m, 1H), 3.02 (s, 6H), 1.80 (m, 3H), 1.57 (m, 3H); ¹³C-NMR (75 MHz, DMSO-d6)™ 187.8, 160.7, 152.5, 145.0, 142.5, 131.2, 130.9, 122.8, 116.8, 116.6, 112.4, 96.22, 62.3, 30.3, 25.3, 19.1; IR (ATR): 2951, 1645, 1599, 1573, 1439, 1400, 1371, 1342, 1287, 1216, 1187, 1161, 1119 cm⁻¹; ESI-MS m/z352.3 [M+H⁺].

4′-hydroxy-4-methoxychalcone (9). 4′-hydroxyacetophenone (6, 1.63 g, 12 mmol) and p-anisaldehyde (8, 1.45 mL, 12 mmol) were dissolved in MeOH (30 mL) in a 70 mL Teflon Milestone MW reaction vessel. A 2 mL aliquot of 50% (w/v) aq. NaOH was added, and the solution was stirred until the reactants had dissolved fully. The reaction vessel was closed tightly and heated with stirring in a Milestone MW reactor from room temperature to 150° C. over 15 min, held at 150° C. for 20 min, and allowed to cool to room temperature over 20 min. The reaction mixture was poured over ca. 30 g of ice and acidified to pH 1.0 with 1.0 N HCl, forming a yellow precipitate. This solid was isolated by filtration and recrystallized from MeOH to afford 900 mg of golden crystals of 9 (30% yield). TLC: R_{f}=0.25 (hexane/EtOAc 3:2); Melting point: 185-188° C.; ¹H-NMR: (300 MHz, DMSO-d6) δ10.35 (brs, 1H), 8.05, 6.89 (AA′XX′, J_{AA′}=J_{XX′}=2.4, J_{AX}=8.6, J_{AX′}=0.2 Hz, 4H), 7.92, 7.00 (AA′XX′, J_{AA′}=J_{XX′}=2.4, J_{AX}=8.6, J_{AX′}=0.2 Hz, 4H), 7.80, 7.60 (AB peak, J=15.5 Hz, 2H); ¹³C-NMR: (75 MHz, DMSO-d6) δ187.7, 162.7, 161.8, 143.3, 131.7, 131.2m 130.0, 128.207, 120.3, 116.0, 115.0, 56.0; IR (ATR): 3200, 2990, 1643, 1602, 1562, 1512, 1430, 1352, 1286, 1223, 1165, 1046 cm⁻¹; ESI-MS: expected, 254.1; observed, m/z 254.8 [M+H⁺].

4-fluoro-4′-hydroxychalcone 4′-hydroxyacetophenone (1.63 g, 12 mmol) and 4-fluorobenzaldehyde (1.28 mL, 12 mmol) were dissolved in 40 ml ethanol in a 100 ml round bottom flask. 50% (w/v) aqueous sodium hydroxide (2 ml) was added and the solution was stirred overnight (approximately 15.5 hours) at room temperature. Approximately 30 g of ice were added to the solution, which was acidified to pH 4 with 1M HCl. The resulting pale yellow precipitate was removed by filtration and recrystallized from a mixture of ethanol and water. 1.53 g. 54% yield. TLC R_{f}=0.30 (20% ethyl acetate in hexane); Melting point: 177-180° C.; ¹H-NMR (300 MHz, DMSO-d6)™10.39 (brs, 1H) 8.06, 6.90 (AA′XX′, J_{AA′}=J_{XX′}=2.4, J_{AX}=8.7, J_{AX′}=0.3 Hz, 4H), 7.93 (m, 2H), 7.95, 7.58 (AB peak, J=15.6 Hz, 2H), 7.28(m, 2H); ¹³C-NMR (75 MHz, DMSO-d6)™ 187.7, 163.9 (d, J=249.4 Hz) 162.9, 142.1, 132.2 (d, J=2.8 Hz) 131.8, 131.6, (d, J=9 Hz), 129.8, 122.7, 116.5 (d, J=22 Hz), 116.0; IR (ATR): 3145, 1647, 1568, 1509, 1441, 1417, 1342, 1286, 1219, 1166, 1157 cm⁻¹; ESI-MS m/z 243.1 [M+H⁺].

4,4′-dimethoxychalcone. In a 100 mL round bottom flask, 4-methoxyacetophenone (1.5 g, 10.0 mmol), p-anisaldehyde (1.63 g, 12.0 mmol), 2.0 mL of 50% aq. NaOH, and 30 mL of MeOH were combined. The solution was closed with a stopper and stirred at room temperature for 8 h. The precipitate was filtered, washed with 2.0 mL of 50% aq. MeOH, and allowed to air dry. 2.29 g of white solid were isolated. 85% yield. TLC R_{f}=0.50 (20% ethyl acetate in hexane); Melting point: 89-90° C.; ¹H-NMR (300 MHz, DMSO-d6)™ 8.16, 7.08 (AA′XX′ peak, J_{AA′}=J_{XX′}=2.4, J_{AX}=8.7, J_{AX′}=0.2 Hz, 4H), 7.88, 7.63 (AB peak, J=15.3 Hz, 2H), 7.85, 7.02 (AA′XX′ peak, J_{AA′}=J_{XX′}=2.7, J_{AX}=8.7, J_{AX′}=0.2 Hz, 4H), 3.86 (s, 3H), 3.82 (s, 3H); ¹³C-NMR (75 MHz, DMSO-d6)™ 187.9, 163.7, 161.9, 143.8, 131.4, 131.3, 128.1, 120.2, 115.1, 114.6, 56.2, 56.0; IR (ATR): 3550, 3491, 1653, 1593, 1568, 1509, 1463, 1441, 1423, 1335, 1294, 1248, 1215, 1180, 1165, cm⁻¹.

2-fluoro-4′-methoxychalcone. Using the above method: 2.07 g; 65% yield; TLC R_{f}=0.56 (20% ethyl acetate in hexane); Melting point: 92-93° C.; ¹H-NMR (300 MHz, DMSO-d6)™ 8.17, 7.10 (AA′XX′ peak, J_{AA′}=J_{XX′}=2.4, J_{AX}=8.7, J_{AX′}=0.3 Hz, 4H), 8.13 (m, 1H), 7.51 (m, 1H), 7.32 (m, 2H), 8.09, 7.72 (AB peak, J=15.7 Hz, 2H), 7.85, 7.02 (AA′XX′ peak, J_{AA′}=J_{XX′}=2.7, J_{AX}=8.7, J_{AX′}=0.2 Hz, 4H), 3.86 (s, 3H), 3.82 (s, 3H); ¹³C-NMR (75 MHz, DMSO-d6)™ 187.9, 164.1, 161.6 (d, J=255 Hz), 134.9 (d, J=4.5 Hz), 133.1 (d, J=8.0 Hz), 131.6 (d, J=7.4 Hz), 130.9, 129.7, 125.6, 124.9, 123.0 (d, J=14.6 Hz), 116.7 (d, J=23.3 Hz), 114.8, 56.2; IR (ATR): 2977, 1656, 1609, 1573, 1484, 1458, 1423, 1337, 1318, 1285, 1257, 1230, 1189, 1094 cm⁻¹.

4-fluoro-4′-methoxychalcone. Using the above method: 2.68 g; 87% yield; TLC R_{f}=0.45 (40% ethyl acetate in hexane); Melting point: 107-108° C.; ¹H-NMR (300 MHz, DMSO-d6)™ 8.13, 7.05 (AA′XX′ peak, J_{AA′}=J_{XX′}=2.5, J_{AX}=8.7, J_{AX′}=0.3 Hz, 4H), 7.93 (m, 2H), 7.87, 7.68 (AB peak, J=15.8 Hz, 2H), 7.26 (m, 2H), 3.81 (s, 3H); ¹³C-NMR (75 MHz, DMSO-d6)™ 88.0, 164 (d, J=248 Hz), 163.9, 142.5, 132.2, 131.7 (d, J=8.6 Hz), 131.6, 131.1, 122.6, 116.5 (d, 22.3 Hz), 114.7, 56.2; IR (ATR): 3322, 1655, 1598, 1572, 1508, 1424, 1339, 1315, 1286, 1218, 1189, 1158 cm−1.

3′,4′,4-trimethoxychalcone Using the above method: 1.91 g; 64% yield; TLC R_{f}=0.17 (20% ethyl acetate in hexane); Melting point: 73-74° C.; ¹H-NMR (300 MHz, DMSO-d6)™ 7.88 (dd, J=8.5, 2.0 Hz, 1H), 7.84, 7.01 (AA′XX′ peak, J_{AA′}=J_{XX′}=2.2, J_{AX}=8.9, J_{AX′}=−0.1 Hz, 4H), 7.80, 7.69 (AB peak, J=15.5 Hz, 2H), 7.60 (d, J=2.0 Hz, 1H), 7.08 (d, J=8.5 Hz, 1H), 3.87 (s, 3H), 3.86 (s, 3H), 3.82 (s, 3H); ¹³C-NMR (75 MHz, DMSO-d6)™187.9, 161.9, 153.7, 149.5, 143.7, 131.4, 131.3, 128.2, 123.8, 120.1, 115.0, 111.5, 111.4, 56.4, 56.3, 56.0; IR (ATR): 3383, 3200, 1649, 1594, 1568, 1513, 1459, 1424, 1336, 1295, 1265, 1248, 1201, 1180, 1163, 1150, 1055, 1027.

3,3′-dimethoxy-4-hydroxychalcone (12). 3-Methoxy4-[(tetrahydro-2H-pyran-2-yl)-oxy]-benzaldehyde (hereafter called THP-vanillin, 500 mg, 2.1 mmol), 3′-methoxyacetophenone (315 mg, 2.1 mmol), NaOH (84 mg, 2.1 mmol), and 5.0 mL of MeOH were combined in a 20 mL round-bottom flask equipped with a magnetic stirring bar and stirred for 12 h at room temperature. Acetyl chloride (0.5 mL, 7.0 mmol) then was added quickly to the flask. After stirring for 10 min, the mixture was poured into 20 mL of 1.0 N HCl and 20 mL of CH₂Cl₂. The solution was extracted twice with CH₂Cl₂. The organic phases were combined and extracted with 40 mL of 1.0 N NaOH. The aq. phase was washed twice with CH₂Cl₂, acidified to pH 1.0 with 1.0 N HCl, and then extracted with 20 mL of CH₂Cl₂ (2×). The organic fractions were combined, dried over MgSO₄, filtered, and concentrated to a brown oil. The oil was purified by flash silica gel chromatography (CH₂Cl₂/EtOAc 4:1) to give 120 mg of 12 as a yellow oil (20% yield). TLC: R_{f}=0.69 (CH₂Cl₂/EtOAc 4:1); ¹H-NMR: (300 MHz, CDCl₃) δ7.78 (d, J=15.6 Hz, 1H), 7.57 (dt, J=7.7, 1.3 Hz, 1H), 7.52 (dd, J=4, 1.3 Hz), 7.37 (t, J=7.9 Hz, 1H), 7.34 (d, J=15.6 Hz, 1H), 7.18 (dd, J=8.2, 2.0 Hz, 1H), 7.10 (bs, 1H), 7.09 (ddd, J=8.2, 2.6, 1.0 Hz, 1H), 6.94 (d, J=8.2 Hz, 1H), 6.30 (s, 1H), 3.90 (s, 3H), 3.84 (s, 3H); ¹³C-NMR: (75 MHz, CDCl₃) δ190.6, 160.1, 148.7, 147.2, 145.6, 140.1, 129.7, 127.6, 123.7, 121.2, 119.9, 119.1, 115.2, 113.2, 110.4, 56.2, 55.7; IR (ATR): 3394, 3055, 2940, 2836, 1657, 1577, 1513, 1487, 1465, 1452, 1431, 1376, 1321, 1200, 1174, 1158, 1123; ESI-MS: expected, 284.1; observed, m/z 285.0 [M+H⁺].

### Example 7

### Solution-Phase Synthesis of Cyanopyridines

3-cyano-4,6-di(4-methoxyphenyl)-2-methylpyridine. In a 250 mL round bottom flask equipped with a reflux condenser, 4,4′-dimethoxychalcone (1.07 g, 4.0 mmol), 3-aminocrotononitrile (328 mg, 4.0 mmol), sodium hydroxide (160 mg, 4.0 mmol), and 160 mL of EtOH were combined. The solution was refluxed for 8 h. The condenser was removed, and the solution was stirred at room temperature under an oxygen atmosphere. After 20 min, a precipitate formed. After 4 h, the precipitate was filtered, washed with 4.0 mL of 50% aq. MeOH, and allowed to air dry. 321 mg of white solid were isolated. 24% yield. TLC R_{f}=0.56 (20% ethyl acetate in hexane); Melting point: 153-154° C.; ¹H-NMR (300 MHz, DMSO-d6)™ 8.19, 7.12 (AA′XX′ peak, J_{AA′}=J_{XX′}=2.6, J_{AX}=8.4, J_{AX′}=0.3 Hz, 4H), 7.88 (s, 1H), 7.68, 7.05 (AA′XX′ peak, J_{AA′}=J_{XX′}=2.6, J_{AX}=8.6, J_{AX′}=0.2 Hz, 4H), 3.83 (s, 3H), 3.82 (s, (s, 3H); ¹³C-NMR (75 MHz, DMSO-d6)™ 209.0, 162.6, 162.0, 161.4, 160.0, 158.4, 154.2, 130.9, 129.9, 129.1, 118.3, 116.9, 114.7, 56.0, 55.8, 24.5; IR (ATR): 2990, 2901, 2217, 1610, 1575, 1445, 1385, 1345, 1305, 1261, 1169, 1113, 1090, 1058 cm⁻¹.

3-cyano-4-(2-fluorophenyl)-6-(4-methoxyphenyl)-2-methylpyridine. Using the above method: 328 mg; 26% yield; TLC R_{f}=0.60 (20% ethyl acetate in hexane); Melting point: 152-153° C.; ¹H-NMR (300 MHz, DMSO-d6)™ 8.20, 7.05 (AA′XX′ peak, J_{AA′}=J_{XX′}=2.6, J_{AX}=8.7, J_{AX′}=0.3 Hz, 4H), 7.98 (s, 1H), 7.62 (m, 2H), 7.42 (m, 2H), 3.82 (s, 3H), 2.78 (s, 3H); ¹³C-NMR (75 MHz, CDCl₃)™ 162.5, 161.9, 159.5 (d, J=249.9 Hz), 158.9, 148.3, 131.9 (d, J=8.2 Hz), 130.9 (d, J=2.3 Hz), 130.3, 129.2, 124.8 (d, J=3.9 Hz), 124.7, 118.1 (d, 2.2 Hz), 117.0, 116.8, 116.5, 144.6, 106.4, 55.6, 24.5; IR (ATR): 3344, 2990, 2901, 2217, 1610, 1585, 1539, 1488, 1444, 1386, 1309, 1261, 1220, 1171, 1066 cm⁻¹.

3-cyano-4-(3,4-dimethoxyphenyl)-6-(4-methoxyphenyl)-2-methylpyridine. Using the above method: 545 mg; 38% yield; TLC R_{f}=0.20 (20% ethyl acetate in hexane); Melting point: 187-188° C.; ¹H-NMR (300 MHz, DMSO-d6)™7.95 (s, 1H), 7.86 (dd, J=8.5, 2.1 Hz, 1H), 7.80 (d, J=2.1 Hz), 7.70, 7.14 (AA′XX′ peak, J_{AA′}=J_{XX′}=2.5, J_{AX}=8.6, J_{AX′}=0.2 Hz, 4H), 7.09 (d, J=8.6 Hz, 1H), 3.86 (s, 3H), 3.85 (s, 3H), 3.84 (s, 3H), 2.75 (s, 3H); ¹³C-NMR (75 MHz, DMSO-d6)™ 162.4, 161.3, 158.4, 153.5, 151.7, 149.6, 130.9, 129.1, 121.5, 117.5, 115.0, 112.4, 111.2, 56.4, 56.3, 56.1, 24.8; IR (ATR): 2990, 2901, 2218, 1612, 1573, 1517, 1445, 1384, 1303, 1268, 1237, 1170, 1114, 1058 cm⁻¹.

3-cyano-4-(4-dimethylaminophenyl)-6-(4-(tetrahydro-2H-pyran-2-yloxy)phenyl)-2-methylpyridine. In a 100 mL round bottom flask equipped with a reflux condenser, 4-dimethylamino-4′-(tetrahydro-2H-pyran-2-yloxy)chalcone (703 mg, 2.0 mmol), 3-aminocrotononitrile (328 mg, 4.0 mmol), sodium hydroxide (80 mg, 2.0 mmol), and 160 mL of EtOH were combined. The solution was refluxed for 29 h. The condenser was removed, and the solution was stirred at room temperature under an oxygen atmosphere for 12 h. The precipitate was filtered and allowed to air dry. 366 mg of white solid were isolated. 44% yield. TLC R_{f}=0.33 (20% ethyl acetate in hexane); Melting point: 148-150° C.; ¹H-NMR (300 MHz, CDCl₃)™8.02, 7.17 (AA′XX′ peak, J_{AA′}=J_{XX′}=2.2, J_{AX}=8.6, J_{AX′}=0.1 Hz, 4H), 7.60, 6.81 (AA′XX′ peak, J_{AA′}=J_{XX′}=2.3, J_{AX}=8.6, J_{AX′}=0.2 Hz, 4H), 7.59 (s, 1H), 5.51 (m, 1H), 3.90 (m, 1H), 3.63 (m, 2H), 3.05 (s, 6H), 2.85 (s, 3H), 2.09 (m, 2H), 1.90 (m, 2H), 1.67 (m, 3H); ¹³C-NMR (75 MHz, CDCl₃)™207.2, 162.7, 158.9, 158.5, 153.8, 151.5, 131.6, 129.6, 128.9, 123.8, 118.4, 116.8, 116.5, 112.1, 104.2. 96.3, 62.2, 40.3, 30.7, 30.5, 30.4, 30.2, 30.0, 29.7, 25.3, 24.5, 18.8; IR (ATR): 3195, 2924, 2221, 1679, 1582, 1529, 1435, 1370, 1310, 1241, 1199, 1180, 1142, 1054, 1033 cm⁻¹; ESI-MS m/z 414.2 [M+H⁺].

3-cyano-4-(4-dimethylaminophenyl)-6-(4-hydroxyphenyl)-2-methylpyridine. In a 4 mL vial, 3-cyano-4-(4-dimethylaminophenyl)-6-(4-(tetrahydro-2H-pyran-2-yloxy)phenyl)-2-methylpyridine (150 mg, 0.36 mmol) was dissolved in 1.0 mL of 20% trifluoroacetic acid in CH₂Cl₂ were combined. The vial was closed with a teflon cap, shaken, and allowed to stand for 5 min. The solution was concentrated to a dark orange oil under a stream of nitrogen gas. Trituration with 0.5 mL of ether produced 100 mg of a dark red solid. 83% yield. TLC R_{f}=0.22 (40% ethyl acetate in hexane); Melting point: 144-145° C.; ¹H-NMR (300 MHz, DMSO)™8.07, 6.87 (AA′XX′ peak, J_{AA′}=J_{XX′}=2.4, J_{AX}=13.6, J_{AX′}=5.1 Hz, 4H), 7.76 (s, 1H), 7.61, 6.84 (AA′XX′ peak, J_{AA′}=J_{XX′}=2.3, J_{AX}=8.9, J_{AX′}=0.1 Hz, 4H), 3.00 (s, 6H), 2.74 (s, 3H); ¹³C-NMR (75 MHz, CDCl₃)™ 162.5, 160.4, 158.3, 153.7, 151.9, 130.2, 129.8, 128.7, 123.7, 118.8, 116.3, 116.2, 112.6, 103.4, 31.3, 24.7; IR (ATR): 3195, 2973, 2230, 1678, 1596, 1582, 1528, 1436, 1371, 1241, 1197, 1180, 1142, 1033 cm⁻¹; ESI-MS m/z 330.0 [M+H⁺].

3-cyano-4-(4-methoxyphenyl)-6-(4-hydroxyphenyl)-2-methylpyridine. 4′-hydroxy4-methoxychalcone (508 mg, 2.0 mmol), 3-aminocrotononitrile (328 mg, 4.0 mmol), sodium hydroxide (80 mg, 2.0 mmol), and 40 mL of EtOH were combined in a 70 mL teflon Milestone microwave reaction vessel. The reaction vessel was tightly closed and heated, with stirring using a Milestone microwave from room temperature to 150° C. over 15 minutes, held at 150° for 20 minutes and allowed to cool to room temperature. The solution was poured into a 100 mL round bottom flask. 4.0 mL of 1.0 N aq. HCl was added. The solution was stirred at room temperature under an oxygen atmosphere for 12 h. The precipitate was filtered and allowed to air dry. 110 mg of white solid were isolated. 9% yield. TLC R_{f}=0.14 (20% ethyl acetate in hexane); Melting point: 220-221° C.; ¹H-NMR (300 MHz, Acetone-d6)™ 8.18, 6.99 (AA′XX′ peak, J_{AA′}=J_{XX′}=2.5, J_{AX}=8.5, J_{AX′}=0.3 Hz, 4H), 7.83 (s, 1H), 7.72, 7.14 (AA′XX′ peak, J_{AA′}=J_{XX′}=2.6, J_{AX}=8.5, J_{AX′}=0.3 Hz, 4H), 3.85 (s, 3H), 2.75 (s, 3H); ¹³C-NMR (75 MHz, DMSO-d6)™ 162.4, 161.2, 160.6, 158.6, 153.2, 130.8, 129.8, 129.1, 128.5, 118.4, 116.8, 116.3, 114.9, 104.1, 56.1, 24.7; IR (ATR): 3195, 2973, 2221, 1610, 1587, 1533, 1517, 1462, 1382, 1303, 1284, 1252, 1176, 1119, 1048, 1033 cm⁻¹; ESI-MS m/z 316.9 [M+H⁺].

### Example 8

### Solution-Phase Synthesis of Deazalumazines

1,3-dimethyl-5-(4-fluorophenyl)-7-(4-hydroxyphenyl)-pyrido[2,3-d]pyrimidine-2,4(1H,3H)-dione. 4′-fluoro4-hydroxychalcone (480 mg, 2.0 mmol), 6-amino-2,3-dimethyluracil (310 mg, 2.0 mmol), potassium hydroxide (112 mg, 2.0 mmol), and 4.0 mL of ethanol were combined in a 20 mL vial. The vial was purged with oxygen, sealed with a teflon cap, and heated at 80° C. in a conventional drying oven for 16 h. The mixture was allowed to cool to room temperature. 2.0 g of ice was added. After stirring for 15 minutes, the mixture was filtered and washed with 1.0 mL of water and 1.0 mL of ethanol. 112.5 mg of yellow solid was obtained. 15% yield. TLC R_{f}=0.28 (40% ethyl acetate in hexane); ¹H-NMR (300 MHz, DMSO-d6)™ 7.97 (d, 8.9 Hz, 2H), 7.39 (m, 2H), 7.32 (s, 1H), 7.21 (m, 2H), 6.60 (d, J=8.9 Hz, 2H), 3.66 (s, 3H), 3.15 (s, 3H); ¹³C-NMR (75 MHz, DMSO-d6)™ 169.4, 162.5 (d, J=244.4 Hz), 160.5, 159.8, 152.7, 152.1, 136.8, 131.0 (d, J=8.2 Hz), 130.1, 122.0, 118.4, 116.3, 114.8 (d, J=21.4 Hz), 104.0, 30.3, 28.5; ESI-MS m/z 378.1 [M+H⁺].

1,3-dimethyl-5-(4-fluorophenyl)-7-(4-methoxyphenyl)-pyrido[2,3-d]pyrimidine-2,4(1H,3H)-dione. Using the above method: 460 mg. 59% yield. TLC R_{f}=0.14 (20% ethyl acetate in hexane); Melting point: 240-243° C.; ¹H-NMR (300 MHz, CDCl₃) δ 8.0, 7.01 (AA′XX′ peak, J_{AA′}=J_{XX′}=2.5, J_{AX}=8.7, J_{AX′}=0.2 Hz, 4H), 7.39 (s, 1H), 7.32 (m, 2H), 7.15 (m, 2H), 3.89 (s, 3H), 3.87 (s, 3H), 3.39 (s, 3H); ¹³C-NMR (75 MHz, CDCl₃)™ 163.2, 162.7, (d, J=246 Hz), 162.0, 160.6, 159.0, 153.9, 151.7 (d, J=22.4 Hz), 135.5 (d, J=2.9 Hz), 129.7 (d, J=8.3 Hz), 129.1, 117.6, 115.0, 114.8, 114.4, 105.8, 55.5, 30.1, 28.4; IR (ATR): 2989, 2896, 1702, 1660, 1600, 1457, 1439, 1422, 1367, 1287, 1263, 1221, 1165, 1086 cm⁻¹.

### Example 9

### Solution-Phase Synthesis of Triarylpyridines and Precursors

1,5-di-(3-methoxyphenyl)-3-(4-hydroxy-3-methoxyphenyl)-1,5-pentanedione (13). THP-vanillin (500 mg, 2.1 mmol, Webb, T. R. Synthesis 1984, 213-214), 3′-methoxyacetophenone (630 mg, 4.2 mmol), and NaOH (168 mg, 4.2 mmol) were ground together in a mortar and pestle for 30 min. The resulting yellow paste was allowed to stand at room temperature for 12 h. The paste then was dissolved in 20 mL of 1.0 N HCl and 20 mL of CH₂Cl₂. The mixture was stirred for 1 h, after which the organic phase was separated. The aqueous phase was extracted two times with CH₂Cl₂. The organic phases were combined, dried over MgSO₄, filtered, and concentrated to give a brown oil. The oil was purified using flash silica gel chromatography (CH₂Cl₂/EtOAc 4:1) to give 30 mg of 13 as a colorless oil (0.5% yield). TLC: R_{f}=0.72 (CH₂Cl₂/EtOAc 4:1); ¹H-NMR: (300 MHz, CDCl₃) δ7.54 (dt, J=8.0, 1.2 Hz, 2H), 7.46 (dd, J=2.5, 1.2 Hz, 2H), 7.35 (t, J=8.0 Hz), 7.09 (ddd, J=8.0, 2.5, 1.2 Hz, 2H), 6.78 (m, 3H), 5.50 (s, 1H), 3.99 (p, J=7.0 Hz, 1H), 3.42, 3.38 (AB component of ABX system, J_{AB}=16.5 Hz, J_{AX}=J_{BX}=7.0 Hz, 2H); ¹³C-NMR: (75 MHz, CDCl₃) δ 198.6, 159.8, 146.4, 144.3, 138.4, 135.7, 129.6, 120.8, 119.6, 119.5, 114.5, 112.3, 110.7, 55.9, 55.4, 45.3, 37.2; IR (ATR): 3727, 3703, 3629, 3595, 3054, 1684, 1597, 1583, 1517, 1486, 1465, 1452, 1430, 1361, 1288, 1210, 1160, 1125 cm⁻¹; ESI-MS: expected, 434.2; observed, m/z 435.1 [M+H⁺].

2,6-di-(3-bromophenyl)-4-(4-hydroxy-3-methoxyphenyl)-pyridine (16l). THP-vanillin (500 mg, 2.1 mmol), 3′-bromoacetophenone (836 mg, 4.2 mmol), and NaOH (168 mg, 4.2 mmol) were ground together in a mortar and pestle for 30 min. The resulting orange paste was allowed to stand at room temperature for 1 h. The paste then was transferred to a 70 mL Teflon MW reaction vessel along with a magnetic stirring bar. A 0.5 mL aliquot of acetic acid was added to the reaction vessel, followed by butanol (5.0 mL) and hydroxylamine hydrogen chloride (510 mg, 7.3 mmol). The reaction vessel was closed tightly and heated with stirring in the Milestone MW reactor from room temperature to 170° C. over 10 min, held at 170° C. for 10 min, and allowed to cool to room temperature over ca. 30 min. A 20 mL portion of water was added to the vessel, and the reaction mixture was stirred for 8 h at room temperature. A white solid gradually formed. This solid was filtered to give 86 mg of triarylpyridine 16l (8% yield). TLC: R_{f}=0.55 (1% AcOH in CH₂Cl₂). ¹H-NMR: (300 MHz, CDCl₃) δ 8.31 (t, J=1.8 Hz, 2H), 8.11 (ddd, J=7.8, 1.5, 1.2 Hz, 2H), 7.81, (s, 2H), 7.59 (ddd, J=8.0, 2.2, 1.1 Hz, 2H), 7.40 (t, J=8.0 Hz, 2H), 7.29 (dd, J=8.3, 1.9 Hz, 1H), 7.20 (d, J=1.9 Hz, 1H), 7.08 (d, J=8.3 Hz, 1H), 5.81 (s, 1H), 4.04 (s, 3H); ¹³C-NMR: (75 MHz, CDCl₃) δ 156.3, 150.8, 147.3, 147.2, 141.7, 132.3, 130.5, 130.3, 125.9, 123.2, 120.9, 117.6, 115.3, 109.6, 56.5; IR (ATR): 3509, 3061, 1601, 1567, 1548, 1518, 1478, 1469, 1442, 1422, 1387, 1369, 1350, 1271, 1241, 1212, 1177, 1120 cm⁻¹; ESI-MS: expected, 509.0; observed, m/z 509.9 [M+H⁺].

2,6-di-(3-methoxyphenyl)-4-(4-hydroxy-3-methoxyphenyl)-pyridine (16r). Vanillin (1.00 g, 6.57 mmol), 3′-methoxyacetophenone (1.97 g, 6.57 mmol), NH₄OAc (2.00 g, 25.9 mmol), and 30 mL of acetic acid were combined in a 70 mL Teflon MW reaction vessel. A magnetic stirring bar was added to the vessel. The vessel was closed tightly and heated with stirring in the Milestone MW reactor from room temperature to 180° C. over 10 min, held at 180° C. for 20 min, and allowed to cool to room temperature over ca. 30 min. The MW heating sequence was repeated (1×). The reaction mixture was concentrated to an oil under reduced pressure. The oil was dissolved in 50 mL of EtOAc and washed with sat. aq. NaHCO₃ (2×), brine, and H₂O. The organic phase was concentrated under reduced pressure to give a brown oil. The oil was purified by flash silica gel column chromatography (1% AcOH in CH₂Cl₂) to give 65 mg of triarylpyridine 16r as a colorless oil (2% yield). TLC: R_{f}=0.26 (1% AcOH in CH₂Cl₂); ¹H-NMR: (300 MHz, CDCl₃) δ 7.81 (s, 2H), 7.79 (dd, J=2.6, 1.4 Hz, 2H), 7.74 (dt, J=8.0, 1.4 Hz, 2H), 7.42 (t, J=8.0 Hz, 2H), 7.29 (dd, J=8.2, 2.0 Hz, 1H), 7.21 (d, J=2.0 Hz, 1H), 7.07 (d, J=8.2 Hz, 1H), 7.00 (dd, J=8.0, 2.6 Hz, 2H), 5.79 (s, 1H), 4.01 (s, 3H), 3.92 (s, 6H); ¹³C-NMR: (75 MHz, CDCl₃) δ160.2, 157.4, 150.3, 147.2, 147.0, 141.4, 131.5, 129.9, 120.8, 119.8, 117.3, 115.2, 114.8, 113.0, 109.7, 56.4, 55.6; IR (ATR): 3727, 3600, 3052, 1599 1583, 1547, 1515, 1492, 1401, 1288, 1169, 1125 cm⁻¹; ESI-MS: expected, 413.2; observed, m/z 414.1 [M+H⁺].

### Example 10

### Spectral Properties of Certain Compounds of this Invention

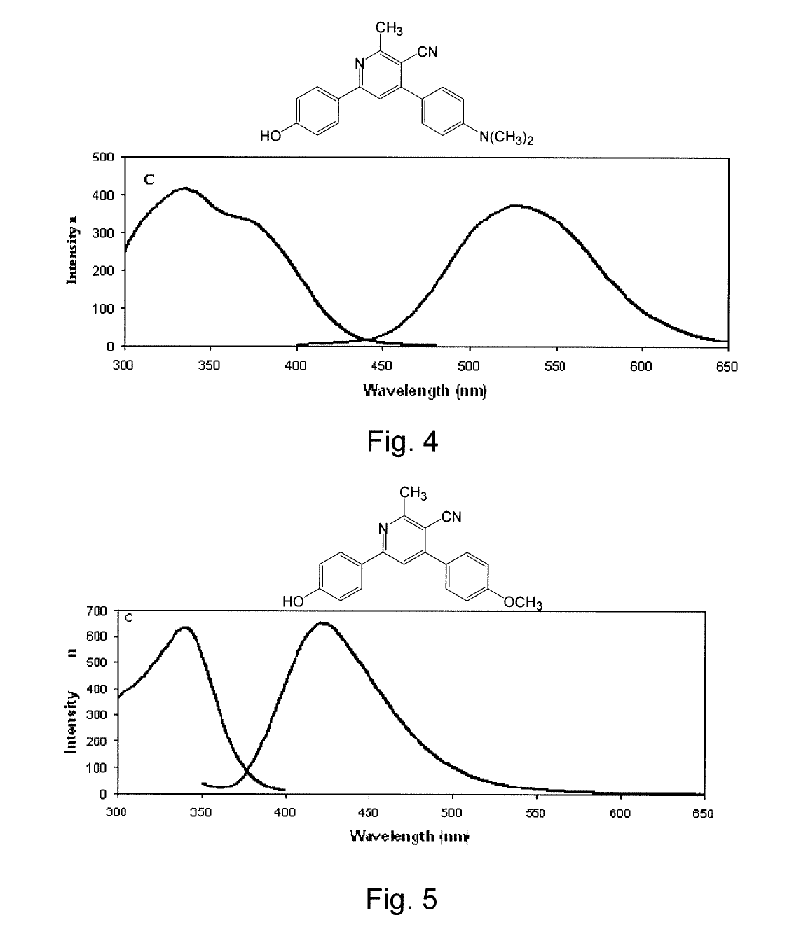

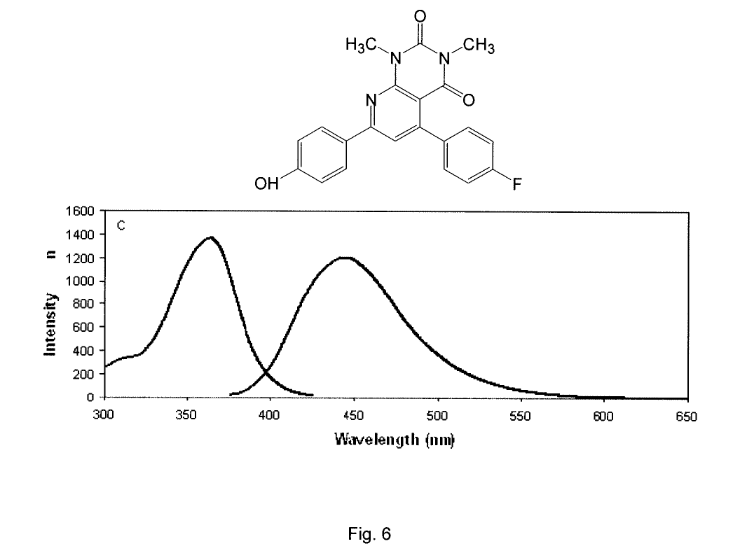

FIG. 4 shows the excitation and emission spectra of purified cyanopyridine 2h obtained from solution-phase synthesis, which is the same as those of the crude product 2h obtained from SPOT-synthesis. FIG. 5 shows the excitation and emission spectra of purified cyanopyridine 2g obtained from solution-phase synthesis, which is the same as those of the crude product 2g obtained from SPOT-synthesis. FIG. 6 shows the excitation and emission spectra of purified deazalumazine 3c obtained from solution-phase synthesis, which is the same as those of the crude product 3c obtained from SPOT-synthesis.

Table 8 reports the influence of pH on the spectral properties of cyanopyridines of formula 2 where R₁ and R₂ are as listed. Coumarin 120 ((φ_{f}=0.88, λ_{exc}=354 nm, λₑₘₘ=435 nm in ethanol was used as an external standard. Error limits=±15%.

Table 9 reports the influence of pH on spectral properties of deazalumazines of formula 3 where R₁ and R₂ are as listed. Coumarin 120 ((φ_{f}=0.88, λ_{exc}=354 nm, λₑₘₘ=435 nm in ethanol) was used as the external standard. Error limits=±15%.

Table 10 reports the influence of solvent on spectral properties of synthesized fluorophores of formulas 2 or 3 where R₁ and R₂ are as listed. External standard: coumarin 120 ((φ_{f}=0.88, λ_{exc}=354 nm, λₑₘₘ=435 nm in ethanol). Error=±15%.

It is understood that the examples and embodiments described herein are for illustrative purposes only and that various modifications or changes in light thereof will be suggested to persons skilled in the art and are to be included within the spirit and purview of this application and scope of the appended claims.

TABLE 8
Influence of pH on the spectral properties of cyanopyridines

| Compound | R¹ | R² | Buffer (in EtOH) | pH | λₑₓ | λₑₘ | φ_{f}^{a} |
|---|---|---|---|---|---|---|---|
| 2 | 4-OMe | 2-F | 1.0 mM TFA | 1.6 | 337 | 419 | 0.64 |
| 2 | 4-OMe | 2-F | 1.0 mM AcOH | 4.8 | 336 | 419 | 0.65 |
| 2 | 4-OMe | 2-F | 1.0 mM NH_{↓}OAc | 7.6 | 336 | 418 | 0.71 |
| 2 | 4-OMe | 2-F | 1.0 mM NH_{↓}OAc/KOH (1:1) | 10.0 | 336 | 420 | 0.68 |
| 2 | 4-OH | 4-OMe | 1.0 mM TFA | 1.6 | 342 | 422 | 0.14 |
| 2 | 4-OH | 4-OMe | 1.0 mM AcOH | 4.8 | 339 | 423 | 0.14 |
| 2 | 4-OH | 4-OMe | 1.0 mM NH_{↓}OAc | 7.6 | 340 | 423 | 0.12 |
| 2 | 4-OH | 4-OMe | 1.0 mM NH_{↓}OAc/KOH (1:1) | 10.0 | 340 | 421 | 0.09 |
| 2 | 4-OMe | 4-OMe | 1.0 mM TFA | 1.6 | 335 | 408 | 0.78 |
| 2 | 4-OMe | 4-OMe | 1.0 mM AcOH | 4.8 | 337 | 407 | 0.76 |
| 2 | 4-OMe | 4-OMe | 1.0 mM NH_{↓}OAc | 7.6 | 337 | 408 | 0.74 |
| 2 | 4-OMe | 4-OMe | 1.0 mM NH_{↓}OAc/KOH (1:1) | 10.0 | 336 | 408 | 0.76 |
| 2 | 4-OH | 4-NMe₂ | 1.0 mM TFA | 1.6 | 336 | 526 | 0.06 |
| 2 | 4-OH | 4-NMe₂ | 1.0 mM AcOH | 4.8 | 336 | 530 | 0.11 |
| 2 | 4-OH | 4-NMe₂ | 1.0 mM NH_{↓}OAc | 7.6 | 335 | 529 | 0.09 |
| 2 | 4-OH | 4-NMe₂ | 1.0 mM NH_{↓}OAc/KOH (1:1) | 10.0 | 336 | 528 | 0.09 |

TABLE 9
Influence of pH on spectral properties of deazalumazines

| Compound | R¹ | R² | Buffer (in EtOH) | pH | λₑₓ | λₑₘ | φ_{f}^{a} |
|---|---|---|---|---|---|---|---|
| 3 | 4-OH | 4-F | 1.0 mM TFA | 1.6 | 362 | 444 | 0.25 |
| 3 | 4-OH | 4-F | 1.0 mM AcOH | 4.8 | 363 | 444 | 0.25 |
| 3 | 4-OH | 4-F | 1.0 mM NH_{↓}OAc | 7.6 | 362 | 443 | 0.20 |
| 3 | 4-OH | 4-F | 1.0 mM NH_{↓}OAc/KOH (1:1) | 10.0 | 362 | 442 | 0.16 |
| 3 | 4-OMe | 4-F | 1.0 mM TFA | 1.6 | 359 | 424 | 0.18 |
| 3 | 4-OMe | 4-F | 1.0 mM AcOH | 4.8 | 360 | 423 | 0.18 |
| 3 | 4-OMe | 4-F | 1.0 mM NH_{↓}OAc | 7.6 | 360 | 425 | 0.20 |
| 3 | 4-OMe | 4-F | 1.0 mM NH_{↓}OAc/KOH (1:1) | 10.0 | 360 | 425 | 0.21 |

TABLE 10
Influence of solvent on spectral properties of synthesized
fluorophores of formulas 2 or 3

| Compound | R¹ | R² | Solvent | λ | λ | φ_{f}^{a} |
|---|---|---|---|---|---|---|
| 2 | 4-OMe | 4-OMe | CHCl₃ | 336 | 393 | 0.74 |
| 2 | 4-OMe | 4-OMe | THF | 337 | 396 | 0.72 |
| 2 | 4-OMe | 4-OMe | DMSO | 340 | 419 | 0.81 |
| 2 | 4-OMe | 4-OMe | EtOH | 335 | 409 | 0.77 |
| 2 | 4-OH | 4-OMe | CHCl₃ | 333 | 404 | 0.66 |
| 2 | 4-OH | 4-OMe | THF | 339 | 405 | 0.58 |
| 2 | 4-OH | 4-OMe | DMSO | 344 | 440 | 0.31 |
| 2 | 4-OH | 4-OMe | EtOH | 341 | 424 | 0.15 |
| 2 | 4-OH | 4-NMe₂ | CHCl₃ | 374 | 471 | 0.53 |
| 2 | 4-OH | 4-NMe₂ | THF | 333 | 486 | 0.53 |
| 2 | 4-OH | 4-NMe₂ | DMSO | 341 | 538 | 0.17 |
| 2 | 4-OH | 4-NMe₂ | EtOH | 334 | 526 | 0.10 |
| 3 | 4-OH | 4-F | CHCl₃ | 361 | 417 | 0.13 |
| 3 | 4-OH | 4-F | THF | 361 | 417 | 0.12 |
| 3 | 4-OH | 4-F | DMSO | 367 | 454 | 0.23 |
| 3 | 4-OH | 4-F | EtOH | 363 | 442 | 0.23 |
| 3 | 4-OMe | 4-F | CHCl₃ | 361 | 408 | 0.12 |
| 3 | 4-OMe | 4-F | THF | 360 | 410 | 0.09 |
| 3 | 4-OMe | 4-F | DMSO | 364 | 429 | 0.20 |
| 3 | 4-OMe | 4-F | EtOH | 361 | 423 | 0.22 |

We claim:

1. A method for identifying fluorescent molecules among a plurality of candidate fluorescent molecules which comprises the steps of:
(a) providing a spatially-addressed array of one or more reactive compounds bound to a unitary substrate;
(b) reacting the array of surface-bound reactive compounds with one or more reactive compounds, reagents or both not bound to the substrate under reaction conditions such that an array of surface-bound spatially-addressable candidate fluorescent molecules is synthesized on the unitary substrate;
(c) illuminating the array of candidate fluorescent molecules on the unitary substrate with light of one or more selected wavelengths to excite fluorescence of the array of candidate fluorescent molecules bound to the unitary substrate; and
(d) measuring the spatial distribution of fluorescence emitted from the array to identify fluorescent molecules in the array.

2. The method of claim 1 wherein the candidate fluorescent molecules can be non-destructively cleaved from the unitary substrate.

3. The method of claim 1 wherein the surface-bound reactive compounds are bound to the surface by a linker.

4. The method of claim 3 wherein the linker is acid-cleavable or photocleavable at a wavelength different from that used to illuminate the array.

5. The method of claim 1 wherein the array is a macroarray or a microarray.

6. The method of claim 1 wherein the surface-bound reactive compounds are arylaldehydes, arylketones, chalcones or triaryldiketones.

7. The method of claim 1 wherein the surface-bound reactive compounds are chalcones and the non-surface-bound reactant, reagent or both is an aminouracil, an amidine, acetonitrile/base, hydrazine hydrate, an aryl hydrazine, an aminocyclohexenone, an aminobenzimidazole, or an isatin-amino acid mixture.

8. The method of claim 1 wherein the surface-bound reactive compounds are chalcones, the non-surface -bound reactant, reagent or both is an aminouracil and the surface-bound candidates are deazalumazines.

9. The method of claim 1 wherein the surface-bound reactive compounds are chalcones, the non-surface-bound reactant, reagent or both is an amidine and the surface-bound candidates are pyrimidines.

10. The method of claim 1 wherein the surface-bound reactive compounds are chalcones, the non-surface-bound reactant, reagent or both is acetonitrile/base and the surface-bound candidates are cyanopyridines.

11. The method of claim 1 wherein the surface-bound reactive compounds are chalcones, the non-surface-bound reactant, reagent or both is hydrazine hydrate and the surface-bound candidates are diaryl pyrazoles.

12. The method of claim 1 wherein the surface-bound reactive compounds are chalcones, the non-surface-bound reactant, reagent or both is an aryl hydrazine and the surface-bound candidates are diaryl N-arylpyrazolines.

13. The method of claim 1 wherein the surface-bound reactive compounds are chalcones, the non-surface-bound reactant, reagent or both is an aminocyclohexenone and the surface-bound candidates are diaryl pyridine derivatives.

14. The method of claim 1 wherein the surface-bound reactive compounds are chalcones, the non-surface-bound reactant, reagent or both is mixture of an isatin and an amino acid and the surface-bound candidates are isatin-chalone condensation products.

15. The method of claim 1 wherein the surface-bound reactive compounds are chalcones, the non-surface-bound reactant, reagent or both is an aminobenzimidazole and the surface-bound candidates are diaryl tetrahydropyrimidines.

16. The method of claim 1 wherein the surface-bound reactive compounds are triaryl 1,5-diketones.

17. The method of claim 16 wherein the non-surface-bound reactant, reagent or both is NH₄OAc/O₂ and the surface-bound candidates are triarylpyridines.

18. The method of claim 17 wherein the surface-bound candidates are non-symmetrical triarylpyridines.

19. The method of claim 1 wherein said step of reacting the array of surface-bound reactive compounds with one or more reactive compounds, reagents or both not bound to the substrate comprises the step of providing microwave electromagnetic radiation to said array of surface-bound reactive compounds during reaction.

20. The method of claim 1 further comprising measuring wavelength distributions of fluorescence emitted by at least a portion of said candidate fluorescent molecules on the unitary substrate.

21. The method of claim 1 wherein said unitary substrate is a planar cellulose substrate derivatized with acid cleavable or photocleavable linkers.

22. A method for characterizing the spectral properties of a plurality of candidate fluorescent molecules which comprises the steps of:
(a) providing a spatially-addressable array of one or more reactive compounds bound to a unitary substrate;
(b) reacting the array of surface-bound reactive compounds with one or more reactive compounds, reagents or both not bound to the substrate under reaction conditions such that an array of surface-bound spatially-addressable candidate fluorescent molecules is synthesized on the unitary substrate;
(c) illuminating the array of candidate fluorescent molecules on the unitary substrate with light of one or more selected wavelengths to excite fluorescence of the array of candidate fluorescent molecules bound to the unitary substrate; and
(d) measuring the spatial distribution of fluorescence emitted from the array to characterize one or more optical properties of candidate fluorescent molecules in the array.

23. The method of claim 22 wherein the optical properties characterized include one or more of excitation wavelength distribution, emission wavelength distribution, Stroke's Shift, quantum yield or photodecomposition yield.

24. The method of claim 22 further comprising the steps of isolating at least one of the candidate fluorescent molecules, releasing it from said unitary substrate and characterizing a spectral property of the released candidate fluorescent molecule.

25. A fluorescent molecule synthesized and identified by the method of claim 1.

26. A method for synthesizing a triarylpyridine which comprises the steps of:
(a) providing a surface-bound triaryl-1,5-dione; and
(b) condensing the surface-bound triaryl-1,5-diketone with NH₄OAc (ammonium acetate) to form a surface-bound triarylpyridine.

27. The method of claim 26 wherein the surface-bound triaryl-1,5-diketone is formed by reaction of a surface-bound chalcone with a non-surface-bound aryl ketone.

28. The method of claim 27 wherein the surface-bound chalcone is formed by reaction of a surface-bound aryl ketone with a non-surface-bound aryl aldehyde.

29. The method of claim 28 wherein the surface-bound aryl ketone, the non-surface bound aryl ketone and the non-surface-bound aryl aldehyde are differently substituted on their aryl rings.

30. The method of claim 28 wherein the aryl ketones and the aryl aldehyde each have at least one substituent on their aryl ring selected from —NO₂, —CN, halogen, alkyl, alkenyl, alkynyl, —OH, —OR, —N(R′)₂ groups wherein R is an alkyl, alkenyl, alkynyl or acyl group and R′ is, independent of other R′, selected from hydrogen, alkyl, alkyenyl, alkynyl or acyl, and wherein the alkyl, alkenyl, alkynyl, or acyl of R and R′ are optionally substituted with one or more halogens, —CN, —NO₂, or —OH groups.

31. The method of claim 26 wherein a spatially addressed array of triarylpyridines is formed on a unitary substrate.

32. An unsymmetrical triarylpyridine synthesized my the method of claim 26.

33. A heterocyclic compound having formula: 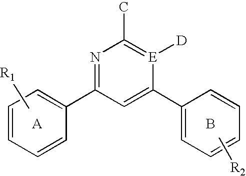
where the A and B rings are aryl rings,
C is an aryl ring having an R₃ substituent, E is carbon and D is a hydrogen or a cyano group;
E is carbon and C and D together form a 5- or 6-member optionally-substituted carbon ring in which one or two ring carbons are replaced with nitrogens; or
E is nitrogen, D is absent and C is an alky or aryl ring;
R₁, R₂ and R₃ represent one or more than one substituent on the indicated ring, wherein each R₁, R₂ and R₃, independently of other R₁, R₂ and R₃ groups, are selected from hydrogen, —NO₂, —CN, halogen, alkyl, alkenyl, alkynyl, phenyl, —OH, —OR, —N(R′)₂ groups wherein R is an alkyl, alkenyl, alkynyl or acyl group and R′ is, independent of other R′, selected from hydrogen, alkyl, alkyenyl, alkynyl or acyl, wherein one of aryl ring A or aryl ring B carry at least one non-hydrogen substituent and wherein the alkyl, alkenyl, alkynyl, acyl of R, R′ or R₁₋₃ are optionally substituted with one or more halogens, —CN, —NO₂, or —OH groups.

34. The pyridine derivative of claim 33 having the formula: 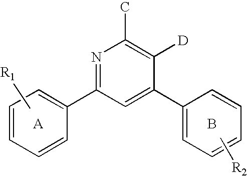
where A and B are aryl rings,
C is an aryl ring having an R₃ substituent and D is a hydrogen or a cyano group or C and D together form a 5- or 6-member optionally-substituted carbon ring in which one or two ring carbons are replaced with nitrogens;
R₁, R₂ and R₃ represent one or more than one substituent on the indicated ring, wherein each R₁, R₂ and R₃, independently of other R₁, R₂ and R₃ groups, are selected from hydrogen, —NO₂, —CN, halogen, alkyl, alkenyl, alkynyl, phenyl, —OH, —OR, —N(R′)₂ groups wherein R is an alkyl, alkenyl, alkynyl or acyl group and R′ is, independent of other R′, selected from hydrogen, alkyl, alkyenyl, alkynyl or acyl, wherein one of aryl ring A or aryl ring B carry at least one non-hydrogen substituent and wherein the alkyl, alkenyl, alkynyl, acyl of R, R′ or R₁₋₃ are optionally substituted with one or more halogens, —CN, —NO₂, or —OH groups.

35. The pyridine derivative of claim 33 wherein one of aryl ring A or aryl ring B is substituted with an —OH group.

36. The pyridine derivative of claim 33 having the formula: 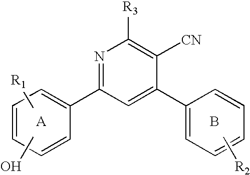
wherein R₃ is an alkyl or phenyl group.

37. The pyridine derivative of claim 36 wherein R₃ is a methyl group.

38. The pyridine derivative of claim 36 wherein the aryl B ring is benzofuranyl.

39. A pyridine derivative of claim 33 having the formula: 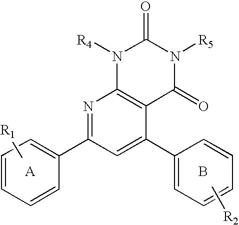
wherein R₄ and R₅, independently, are selected from alkyl groups having 1-6 carbon atoms.

40. The pyridine derivative of claim 39 wherein the aryl B ring is benzofuranyl.

41. A compound according to claim 33 having the formula: 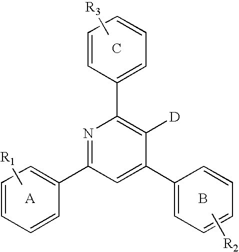
wherein the A, B and C rings are aryl rings;
D is hydrogen or a —CN group;
R₁, R₂ and R₃ represent one or more than one substituent on the indicated ring, wherein each R₁, R₂ and R₃, independently of other R₁, R₂ and R₃ groups, are selected from hydrogen, —NO₂, —CN, halogen, alkyl, alkenyl, alkynyl, aryl, arylalkyl, —OH, —OR, heterocyclic, heteroaryl, and —N(R′)₂ groups wherein R is an alkyl, alkenyl, alkynyl, aryl, arylalkyl, heterocyclic, or heteroaryl group and R′ is, independent of other R′, selected from hydrogen, alkyl, aryl, arylalkyl, heterocyclic, or heteroaryl group wherein each of aryl ring A and aryl ring B carry at least one non-hydrogen substituent and wherein the alkyl, alkenyl, alkynyl, arylalkyl, heterocyclic, or heteroaryl groups of R, R′ or R₁₋₃ are optionally substituted with one or more halogens, —CN, —NO₂, alkyl, alkenyl, alkynyl, or aryl groups.

42. The compound of claim 41 wherein each of phenyl rings A, B and C is differently substituted.

43. A pyrimidine compound of claim 33 having the formula: 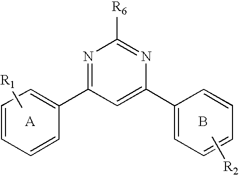
wherein R₆ is selected from alkyl, aryl or —NRR″ where R and R″ are independently selected from hydrogen, alkyl, or aryl.
